(19) United States
(12) Patent Application Publication
(10) Pub. No.: US 2021/0140953 A1
(43) Pub. Date: May 13, 2021

(54) EFFICIENT LIGAND EXCHANGE OF A DETERGENT BILAYER ON THE SURFACE OF METAL NANOPARTICLES FOR MOLECULAR FUNCTIONALIZATION AND ASSEMBLY, CORRESPONDING FUNCTIONALIZED NANOPARTICLES AND NANOPARTICLE ASSEMBLIES, AND THEIR USE IN PLASMONIC APPLICATIONS INCLUDING SURFACE-ENHANCED RAMAN SPECTROSCOPY

(71) Applicant: Universität Duisburg-Essen, Essen (DE)

(72) Inventors: Matthias KÖNIG, Osnabrück (DE); Jun Hee YOON, Essen (DE); Sebastian SCHLÜCKER, Velbert (DE); Florian SELBACH, Gräfelfing (DE)

(21) Appl. No.: 16/492,467

(22) PCT Filed: Mar. 9, 2018
(86) PCT No.: PCT/EP2018/055963

§ 371 Date: Sep. 9, 2019

Related U.S. Application Data
(60) Provisional application No. 62/469,764, filed on Mar. 10, 2017.

(51) Int. Cl. G01N 33/543; G01N 21/65

(52) U.S. Cl. CPC G01N 33/54346; B82Y 30/00; G01N 21/658

(57) ABSTRACT

The present invention relates to a method allowing a particularly efficient ligand exchange of a detergent bilayer on the surface of metal nanoparticles for molecular functionalization and assembly, and corresponding functionalized nanoparticles and nanoparticle assemblies that can be prepared using this method, as illustrated in FIG. 16, as well as their use, e.g., for plasmonic applications such as surface-enhanced Raman scattering (SERS). In particular, the invention provides corresponding methods for preparing a dimeric nanoparticle assembly, a core-satellite nanoparticle assembly, and a functionalized nanoparticle, respectively.

The present invention relates to a method allowing a particularly efficient ligand exchange of a detergent bilayer on the surface of metal nanoparticles for molecular functionalization and assembly, and corresponding functionalized nanoparticles and nanoparticle assemblies that can be prepared using this method, as illustrated in FIG. 16, as well as their use, e.g., for plasmonic applications such as surface-enhanced Raman scattering (SERS). In particular, the invention provides corresponding methods for preparing a dimeric nanoparticle assembly, a core-satellite nanoparticle assembly, and a functionalized nanoparticle, respectively.

Fluorescence microscopy and fluorescence spectroscopy are among the most widely used optical techniques for the detection of labelled (bio)molecules. The use of fluorophores as external markers has been known for a long time. More recently, quantum dots (QDs)—semiconductor nanocrystals with intense and controlled fluorescence emission—are among the most promising nanostructures for applications not only in the life sciences. Diagnostic 25 applications of QDs include the multiplexed, i.e. parallel, detection of a variety of target molecules. Important areas are the detection of proteins in immunoassays, the detection of neurotransmitters and cellular imaging, see Azzazy (2006) Clinical Chemistry 52, 1238; Jain (2005) Clinica Chimica Acta 358, 37; Rosi (2005) Chemical Reviews 105, 1547. A disadvantage of QDs is the toxicity of the semiconductor material, because compounds such as CdSe, InP/InAs or PbS/PbSe are employed. Quantum dots are well suited as labels in multiplexed applications, i.e. the parallel detection of several target molecules. The number of simultaneously detectable QDs is approximately 3 to 10, which is a significant improvement compared with conventional (organic) fluorophores. Additionally, QDs also possess a much higher photostability compared with conventional fluorophores.

In the life sciences, Raman spectroscopy is currently much less employed in comparison with fluorescence spectroscopy. Recent technological developments (UV/NIR lasers, high-throughput spectrometers, notch filters, CCD cameras) have contributed to an increased use of Raman spectroscopy and microscopy however, the small differential Raman scattering cross sections of most biological materials—resulting in weak Raman signals—is in many cases disadvantageous. By placing molecules close to metallic nanostructures, the Raman scattering signal can be enhanced by up to 14 orders of magnitude. This type of Raman scattering, which is called surface-enhanced Raman scattering (SERS), has therefore a very high sensitivity. In contrast to fluorescence spectroscopy, photo bleaching of the illuminated substrate is generally not a problem in Raman spectroscopy, because the laser light is inelastically scattered (and, in the absence of electronic resonances, not absorbed). The occurrence of tissue autofluorescence as a competing process, for example, can be minimized by near-Infrared (NIR) excitation; autofluorescence can significantly contribute to a decrease in the optical image contrast in fluorescence microscopy, in which excitation in the visible spectral region (Vis) is usually employed.

The most fundamental difference between Raman (vibrational transitions) and fluorescence (electronic transitions) based detection schemes is their intrinsic potential for a multiplexed detection. Raman/SERS approaches have a significantly higher capacity for multiplexing because the line width of Raman bands is approximately 100 times or more smaller as compared to fluorescence emission bands.

The spectral signature of each Raman marker can be presented as a barcode: wavenumbers of Raman bands are encoded in horizontal line positions, whereas the corresponding intensities are encoded in the width of the line. Multiplexing with Raman/SERS marker implies that many different barcodes are detectable within the same spectral window without or only minimal spectral interferences. Each spectrum or barcode must unambiguously be assigned to the corresponding Raman/SERS marker. If the spectral contributions of different markers start to spectrally overlap, mathematical techniques for signal decomposition have to be applied. Besides simple decomposition approaches, also more elaborate methods such as multivariate analysis and chemometric techniques can or must be used.

By conjugating Raman markers to antibodies and metallic nanoparticles for SERS, proteins can be detected at very low concentrations, for example, at the femtomolar level; see Rohr (1989) Anal Biochem 182, 388; Dou (1997) Anal Chem 69, 1492; Ni (1999) Anal Chem 71, 4903; Grubisha (2003) Anal Chem 75, 5936; Xu (2004) Analyst 129, 63. The concept of this SERS-immunoassay is illustrated in FIG. 2 of U.S. Pat. No. 8,854,617: antigens are detected by the characteristic Raman scattering signal of Raman markers which are covalently attached to an antibody (for biological specificity) and to a nanoparticle (for SERS). The specific interaction between antigen and antibody is used both for immobilizing antigens on the gold coated surface and for capturing the antigen from the solution. Because of the distance dependence of SERS, only Raman bands of the Raman marker, which is close to the gold surface, are selectively enhanced; Raman bands of groups which are further distant from the nanoparticle surface, such as the amide bands of the antibody, are not observed. In addition to immunoassays, imaging of target molecules is a further important application. For example, the first demonstration of this Raman technique has been shown by localizing prostate-specific antigen in the epithellum of prostate tissue section (DE 10 2006 000 775; Schlücker (2006) Journal Raman Spectroscopy, 37, 719). These experiments are the proof of principle of SERS microscopy, μSERS, immuno-Raman microspectroscopy, or immuno-SERS microscopy (ISERS mIcroscopy).

Various types of Raman/SERS markers and functionalized nanoparticles are known and are described, e.g., in WO 2004/007767, U.S. Pat. No. 8,854,617, US 200310211488, US 2004/0086897, US 2003/0166297, US 2006/0054506, US 2005/0089901, US 2016/0266104 as well as in: Cao (2002) Science 297, 1536; Cao (2003) J Am Chem Soc 125, 14676; Mulvaney (2003) Langmuir 19, 4784; Ni (1999) Anal Chem 71, 4903; Grubsha (2003) Anal Chem 75, 5936; Yu (2007) Bloconjugate Chem 18, 1155; KIm (2006) Anal Chem 78, 6967; Jun (2007) J Comb Chem 9, 237; Na Li, Functionalization of gold nanoprticles for biomedical and catalytic applications, Université de Bordeaux, 2014; Piasmonics 2011, 6, 113; Interface Focus 2013, 3, 20120092; Nat. Mater. 2010, 9, 60; ACS Nano 2012, 6, 9574; ACS Nano 2014, 8, 8554; ACS Appl. Mater. Interfaces 2016, 8, 20522; J. Phys. Chem. C 2015, 119, 7873; or Adv. Opt. Mater. 2014, 2, 65.

A pair of two spherical nanoparticles (NPs), a dimer, has been a valuable model to study surface plasmon (SP) coupling due to its structural simplicity like a diatomic molecule (Sheikholeslami et al., Nano Left. 2010, 10, 2655-2660). According to the plasmon hybridization model analogous to molecular orbital theory, a symmetric dimer allows just one bright mode when the linearly polarized light is applied to the dimer axis parallel or perpendicular (Nordlander et al., Nano Lett. 2004, 4, 899-903). It implies that the use of dimers greatly reduces the complexity and difficulty in the result interpretation. This has encouraged both theorists and experimentalists to prefer dimers. However, the intrinsic structural non-ideality of experimental dimers constructed by irregular gap distances and polyhedral NPs has disrupted the accurate comparison between theoretical and experimental results (Popp et al., Small 2016, 12, 1667-1675). For this reason, researchers have made their efforts to enhance the ideality of experimental dimers by discarding the variations either in the building block or the gap distance (Tian et al., J. Phys. Chem. C 2014, 118, 13801-13808; Cha et al., ACS Nano 2014, 8, 8554-8563; Ciraci et al., Science 2012, 337, 1072-1074). However, in spite of the reduced non-ideality, such partially idealized dimers are not appropriate for precision plasmonics owing to inevitably quite broad spectral deviations at the single-NP level. In particular, various gap morphologies that are unavoidably created in dimers composed of polyhedral NPs produce disparate SP coupling energies largely deviated from the simulation results, albeit with similar gap distances (Popp et al., Small 2016, 12, 1667-1675).

It is an object of the present invention to provide novel and/or improved functionalized nanoparticles and nanoparticle assemblies, which can advantageously be used for plasmonic applications, including surface plasmon resonance spectroscopy, such as, e.g., surface-enhanced Raman scattering/spectroscopy (SERS).

The nanoparticle assemblies and functionalized nanoparticles provided in accordance with the present invention can be prepared as illustrated in the general scheme in FIG. 16. The respective methods of preparation according to the present invention all make use of a novel approach for the efficient removal of a detergent bilayer (e.g., a CTA⁺ bilayer) from the surface of metal nanoparticles which may be fixed on substrate (e.g., a glass substrate) or may be dispersed in a solvent. This approach as highly advantageous as it allows the subsequent molecular functionalization and/or assembly, as also shown in FIG. 16.

The CTA⁺ bilayer on the nanoparticle (NP) shown in the center of the scheme in FIG. 16 makes NPs positively charged, so that colloidal NPs do not aggregate due to the electrostatic repulsion between NPs. However, the too strong structural robustness of the CTA⁺ bilayer has restricted the functionalization of NPs with other useful ligands. So far, previously developed methods to exchange the CTA⁺ bilayer to another ligands demand harsh conditions and too much time.

The present invention provides novel and/or improved methods for the efficient CTA⁺ bilayer exchange in mild condition (organic solvent+salt+igand), as described in more detail further below. This new method can advantageously be applied to obtain, e.g., DNA-functionalized NPs and various types of assembly structures for study and applications.

The corresponding ligand exchange and assembly are further described in the following, with reference to the steps described and illustrated in FIG. 16.

1) NP Assembly on Substrate (See FIG. 16)

Step (a)—NP1 adsorption on substrate—NP1 covered by the CTA⁺ bilayer adsorbs onto negatively charged substrate by electrostatic attraction. Here, it is important to stay in the appropriate concentration of CTA⁺ molecules in NP1 solution.

Step (b)—Removing the CTA⁺ bilayer—Combination of organic solvent and NaX (NaBr or NaCl) efficiently removes or destabilizes the CTA⁺ bilayer on NP1. This condition cannot touch the CTA⁺ bilayer located in between the NP1 and substrate due to the steric hindrance.

Step (c)—Linker self-assembled monolayer (SAM) formation on NP1—(Di)thiolated linker molecules easily form SAM on the nearly naked surface area of NP1.

Step (d)—Attachment of NP2 on NP1-NP2 dispersed in a mixture of organic solvent and NaX keeps its stability due to degraded but partially existing CTA⁺ molecules on NP2. However, when this NP2 bumps into the thiol group of the linkers on NP1, the existing CTA⁺ molecules on NP2 are easily replaced by the formation of Au—S bond. This NP2 does not adsorb on substrate because the substrate loses its negative charge in such solvent condition.

Step (f)—Stabilizer functionaization on NP2—in order to keep the stability of assemblies after desorption, charged molecules (e.g., MUTAB or MUA; 11-Mercaptoundecanoic acid) must be placed on NP2 before desorption. Stabilizer formation proceeds in a mixture of organic solvent and NaX.

Step (g)—Desorption of assemblies from substrate—Sonication induces desorption of assemblies from substrate. The nearly naked area of NP1, exposed after sonication, is filled with MUTAB that is additionally added in small amount. Here, NaX is not essential because the remained CTA⁺ molecules are highly disordered state on the nearly naked area of NP1.

The assembly process described in 1) above can be expanded to get other types of assemblies by changing the shape, size, or composition of NPs. There are a lot of possibilities and in a merely exemplary manner “cube dimers”, “asymmetric sphere core-sphere satellite”, and “asymmetric cube core-sphere satellite” are described in more detail further below and in the appended examples.

2) NP Assembly in Suspension (See FIG. 16)

Step (a′)—Ligand exchange in suspension—This step is similar to step (d). Thiolated molecules easily replace the highly disordered CTA⁺ bilayer on NPs in the existence of organic solvent and NaX. Here, we use charged thiolated molecules (i.e., MUTAB) to keep the NP stability during ligand exchange.

Step (b′)—Assembly using the electrostatic attraction—Negatively charged NPs like citrate-capped AuNPs electrostatically approach to MUTAB-functionalized NPs. To avoid the aggregation during assembly, the ratio of adding satellites must be much higher than that of core. In other words, core must be fully covered by negatively charged satellites so that the core does not interact with satellites on other cores. The CTA⁺ bilayer does not work like MUTAB because MUTAB cannot escape from core NP whereas CTA⁺ molecules go in and out from the CTA⁺ bilayer. Thus, although satellites bind on the CTA⁺ bilayer on core NP, approached satellites immediately leave the core NP.

The assembly process shown in 2) above can be expanded to get other types of assemblies by changing the shape, size, or composition of NPs. There are a lot of possibilities, and in a merely exemplary manner “satellite-size-controlled symmetric sphere core-sphere satellite” are described in more detail further below and in the examples.

3) DNA-Functionalization Through the CTA⁺ Bilayer Exchange (See FIG. 16)

Since the CTA⁺ bilayer is removed or largely destroyed under a mixture of organic solvent and NaX, other ligands can be introduced on NPs capped by the CTA⁺ bilayer. DNA, as one example of a biomolecule to be introduced, is valuable for bio-application. For making NPs versatile in bio-application, NPs need to be functionalized with DNA. Mirkin's group has developed DNA-functionalization method (Cutler, J. I.; Auyeung, E.; Mirkin, C. A. J. Am. Chem. Soc. 2012, 134, 1376-1391). In accordance with the present invention, DNA-functionalized NPs can be prepared through a novel method. Instead of functionalizing NP1 with dithiol molecules (c) it is also possible to use thiol molecules e.g. HS-DNA shown in (c′). This mono thiol functionized NP1 can also be desorbed by sonication from the substrate (d′).

All these NP systems can be used in plasmonic applications like surface-enhanced spectroscopy such as surface-enhanced Raman spectroscopy and surface-enhanced fluorescence spectroscopy (Acuna, G P.; Möller, F. M.; Holzmeister, P.; Beater, S.; Lalkens, B.; Tinnefeld, P Science 2012, 338, 506-510), optical imaging techniques (Huang, X.; EI-Sayed, I. H.; Qian, W.; EI-Sayed, M. A. J. Am. Chem. Soc. 2006, 128, 2115-2120), photothermal theraphy (Nam, J.; Won, N.; Jin, H.; Chung, H.; Kim, S. J. Am. Chem. Soc. 2009, 131, 13639-13645), and as catalysts (Xie, W.; Schlicker, S. Nat Commun. 2015, 6, 7570).

In accordance with the general scheme shown in FIG. 16 and the corresponding illustrative embodiments outlined above, the present invention will be further described in the following.

In a first aspect, the present invention provides a method of preparing a dimeric nanoparticle assembly, the method comprising:
(i) contacting a first metal nanoparticle (NP1), having a bilayer of a long-chained cationic quaternary ammonium compound bound to its surface, with a negatively charged substrate to obtain NP1 bound to the surface of the negatively charged substrate;
(ii) subjecting NP1, which is bound to the surface of the negatively charged substrate via the bilayer of the long-chained cationic quaternary ammonium compound, to an alkali metal or alkaline earth metal halide and a polar organic solvent to remove the bilayer of the long-chained cationic quaternary ammonium compound from those parts of the surface of NP1 that are not bound to the surface of the negatively charged substrate;
(iii) subjecting NP1, which is bound to the surface of the negatively charged substrate via the bilayer of the long-chained cationic quaternary ammonium compound, to a compound HS—R—X and a polar organic solvent to allow the formation of a self-assembled monolayer of the compound HS—R—X on those parts of the surface of NP1 that are not bound to the negatively charged substrate;
(iv) contacting NP1, which is bound to the surface of the negatively charged substrate via the bilayer of the long-chained cationic quaternary ammonium compound and which has a self-assembled monolayer of the compound HS—R—X bound to those parts of its surface that are not bound to the negatively charged substrate, with a polar organic solvent, an alkali metal or alkaline earth metal halide and a second metal nanoparticle (NP2), wherein NP2 has a bilayer of a long-chained cationic quaternary ammonium compound bound to its surface, to obtain a conjugate of NP1 and NP2, wherein NP1 and NP2 are linked together in said conjugate via a part of the self-assembled monolayer of the compound HS—R—X, said part being bound to the metal surface of both NP1 and NP2, wherein said conjugate of NP1 and NP2 is bound to the surface of the negatively charged substrate via the bilayer of the long-chained cationic quaternary ammonium compound that is bound to the surface of NP1; and
(v) subjecting the conjugate of NP1 and NP2, which is bound to the surface of the negatively charged substrate via the bilayer of the long-chained cationic quaternary ammonium compound that is bound to the surface of NP1, and which has a bilayer of the long-chained cationic quaternary ammonium compound bound to the surface of NP2, to a compound containing an N,N,N-trialkylammonium group and/or a thiol group, an alkali metal or alkaline earth metal halide and a polar organic solvent to remove the bilayer of the respective long-chained cationic quaternary ammonium compound from both NP1 and NP2, allow the formation of a self-assembled monolayer of the compound containing an N,N,N-trialkylammonium group and/or a thiol group on those parts of the surface of both NP1 and NP2 that are not bound by the self-assembled monolayer of the compound HS—R—X, and release the conjugate of NP1 and NP2 from the surface of the negatively charged substrate to provide the dimeric nanoparticle assembly,
   wherein the dimeric nanoparticle assembly thus obtained comprises NP1 and NP2, wherein NP1 comprised in the dimeric nanoparticle assembly has a self-assembled monolayer of the compound containing an N,N,N-trialkylammonium group and/or a thiol group bound to one part of its surface and a self-assembled monolayer of the compound HS—R—X bound to the remaining part of its surface, wherein NP1 and NP2 are linked together via a part of the self-assembled monolayer of the compound HS—R—X, which part is bound to the surface of both NP1 and NP2, and wherein NP2 comprised in the dimeric nanoparticle assembly has a self-assembled monolayer of the compound containing an N,N,N-trialkylammonium group and/or a thiol group bound to the part of its surface that is not bound by the self-assembled monolayer of the compound HS—R—X.

In step (i) of the method according to the first aspect of the invention, a first metal nanoparticle (NP1), having a bilayer of a long-chained cationic quaternary ammonium compound bound to its surface, is contacted with a negatively charged substrate to obtain NP1 bound to the surface of the negatively charged substrate.

The long-chained cationic quaternary ammonium compound which is bound to the surface of the first metal nanoparticle (NP1) is not particularly limited, and in principle any cationic quaternary ammonium compound having at least one long chain (e.g., at least one Ca-s alkyl) attached to the nitrogen atom of the ammonium group can be used. Preferably, the long-chained cationic quaternary ammonium compound that is bound to the surface of NP1 is an (N,N,N-trialkyl)alkylammonium compound, wherein one or two of the alkyl groups comprised in the (N,N,N-trialkyl)-moiety of said (N,N,N-trialkyl)alkylammonium compound are each optionally replaced by a phenyl group, or an alkylpyridinium compound. More preferably, the long-chained cationic quaternary ammonium compound that is bound to the surface of NP1 is an (N,N,N-trialkyl)alkylammonium compound or an alkylpyridinium compound, even more preferably an (N,N,N-trialkyl)alkylammonium compound. The (N,N,N-trialkyl)alkylammonium compound is preferably an (N,N,N-tri(C₁₋₄ alkyl))alkylammonium compound, more preferably a compound (C₈₋₂₂ alkyl)-N(C₁₋₄ alkyl), even more preferably a compound (C₂ alkyl)-N⁺(CH)₃, yet even more preferably a compound H₃C—(CH₂)₇₋₂₁—N⁺(CH₃)₃, and most preferably a compound H₃C—(CH₂)₁₅—N⁺(CH₃)₃. The alkylpyridinum compound is preferably a (Ca& alkyl)-pyridinium compound, more preferably a H₃C—(CH₂)₇₋₂₁-pyridinium compound, and most preferably a compound 1-hexadecylpyridnium. It is thus particularly preferred that the long-chained cationic quaternary ammonium compound that is bound to the surface of NP1 is an (N,N,N-trialkyl)alkylammonium compound or an alkylpyridinium compound, more preferably a compound (C₈₋₂₂ alkyl)-N⁺(C₁₋₄ alkyl)₃, a compound (C₈₋₂₂ alkyl)-N⁺(C₁₋₄ alkyl)₂(phenyl) or a (C₈₋₂₂ alkyl)-pyridinium compound, even more preferably a compound (C₈₋₂₂ alkyl)-N⁺(CH₃)₃, a compound (C₈₋₂₂ alkyl)-N*(CH₃)₂(phenyl) or a (C₈₋₂₂ alkyl)-pyridinium compound, yet even more preferably a compound H₃C—(CH₂)₇₋₂₁—N⁺(CH₃)₃ or a H₃C—(CH₂)₇₋₂₁-pyridinium compound (e.g., a 1-hexadecylpyridinium compound), still more preferably a compound H₃C—(CH₂)₇₋₂₁—N⁺(CH₃)₃, and most preferably a compound H₃C—(CH₂)₁₅—N⁺(CH₃)₃ (which is also referred to herein as cetytrimethylammonium or CTA⁺).

It will be understood that the long-chained cationic quaternary ammonium compound (including any of the corresponding specific compounds described herein) can be associated with any suitable counter anion, e.g., a halide/halogenide counter anion, such as bromide or chloride. For example, the compound H₃C—(CH₂)₁₅—N⁺(CH₃)₃ may be associated with bromide (corresponding to cetyltrimethylammonium bromide, i.e. CTAB) or with chloride (corresponding to cetyltrimethylammonium chloride, i.e. CTAC), and the compound 1-hexadecylpyrldinium may likewise be associated with chloride (corresponding to cetylpyridinium chloride, i.e. CPC) or bromide (corresponding to cetylpyridinium bromide, CPB). The corresponding counter anion does not form part of the bilayer of the long-chained cationic quaternary ammonium compound as such. This bilayer of the long-chained cationic quaternary ammonium compound is also referred to herein as a self-assembled bilayer of the long-chained cationic quaternary ammonium compound.

Specific preferred examples of the long-chained cationic quaternary ammonium compound that is bound to the surface of NP1 are shown in the following table. While these compounds have the general structure (head)-N⁺-(tail), it will be understood that in the case of CPC, the ammonium nitrogen atom (N) forms part of the head group indicated in the table below. Moreover, the table also shows exemplary (non-limiting) counter anions of the various exemplary long-chained cationic quaternary ammonium compounds.

| Compound name | Head | Tail | Counter anion |
|---|---|---|---|
| CTAB | Trimethyl | —(CH₂)₁₆—H | Br⁻ |
| CTAC | Trimethyl | —(CH₂)₁₆—H | Cl⁻ |
| CPC | [chemical structure] | —(CH₂)₁₆—H | Cl⁻ |
| BDAC | —CH₃ | —(CH₂)₁₆—H | Cl⁻ |
| | —C₆H₅ | | |
| | —CH₃ | | |
| DTAB | Trimethyl | —(CH₂)₁₂—H | Br⁻ |
| TTAB | Trimethyl | —(CH₂)₁₄—H | Br⁻ |
| CDAB | —CH₃ | —(CH₂)₁₆—H | Br⁻ |
| | —(CH₂)₂—H | | |
| | —CH₃ | | |

The first metal nanoparticle (NP1) having a bilayer of a long-chained cationic quaternary ammonium compound bound to its surface, which is to be used in step (i) of the method according to the first aspect of the invention, can be prepared in accordance with or in analogy to the corresponding protocol described in the examples section.

In particular, the first metal nanoparticle can be subjected to chemical etching before it is used in step (i). Etching is a mild oxidation process of the surface atoms of the nanoparticle. Corresponding chemical etching procedures are known in the art and are described, e.g., in Ruan, Q et al., Adv. Opt Mater. 2014, 2, 65-73. A chemical etching step is advantageous as it allows to obtain very round and homogenous particles although it is not necessary to conduct chemical etching (thus, round nanoparticles prepared without etching, e.g., in accordance with Part. Pad. Syst. Charact. 2014, 31, 266-273 can also be used). The chemical etching can be conducted as described in Ruan et al., 2014 or as described in the examples.

It is preferred that step (i) is conducted in an aqueous solution of the long-chained cationic quaternary ammonium compound, wherein the concentration of the long-chained cationic quaternary ammonium compound in said aqueous solution is preferably about 1.5 μM to about 10 μM.

The metal nanoparticles to be used in accordance with the present invention, including in particular the first metal nanoparticle (NP1) and the second metal nanoparticle (NP2), will be described in more detail in the following. Any reference to a/the metal nanoparticle or a/the nanoparticle is to be understood as relating to both NP1 and NP2, including specifically to NP1 and/or specifically to NP2.

The metal nanoparticle, such as NP1 and/or NP2, may be a single particle or may comprise a plurality of particles, i.e. an assembly of particles, wherein the single particle and the plurality of particles constitute a nanoparticle. The term “nanoparticle” in the context of the present invention means a particle which preferably has a size (spherical particles: diameter; otherwise: length) of about 1 nm to about 400 nm, more preferably of about 5 nm to about 200 nm, even more preferably of about 10 nm to about 120 nm, and most preferably from about 20 nm to about 100 nm. The assembly of nanoparticles may, for example, comprise at least 2, 3, 5, 10, 15 or 20 nanoparticles. The use of single nanoparticles can be preferred in the case of imaging applications since single nanoparticles may be advantageous in terms of high spatial resolution and multiplexing due to their smaller size as compared to large assemblies of nanoparticles. The nanoparticle of a SERS marker for use in imaging applications preferably has a size of about 1 nm to about 400 nm, more preferably of about 5 nm to about 200 nm, even more preferably of about 10 nm to about 120 nm, and most preferably from about 20 nm to about 100 nm. An assembly of nanoparticles, on the other hand, may exhibit enormous SERS or SERRS enhancements (e.g. for molecules at the junctions of the nanoparticles) upon plasmon excitation. Thus, the use of assemblies of nanoparticles can be preferred when high sensitivities are desired. An assembly of nanoparticles can, for example, be prepared chemically. Examples are micro/nanoemulsions, solid-phase supported chemistry, and template-based approaches. Alternatively, the assemblies can be prepared mechanically, for example by nanomanipulation. Such methods are known to persons skilled in the art and are described, for example, in Baur, Nanotechnology (1998) 9, 360; Worden, Chemistry of Materials (2004) 16, 3746; Zoldesi, Advanced Materials (2005) 17, 924; and Kim, Analytical Chemistry (2006) 78, 6967.

It is preferred that the nanoparticles have a uniform (relatively monodisperse) size distribution. In the context of this invention, the term “uniform size distribution” means that the relative standard deviation with respect to the average size of nanoparticles employed herein is less than 50%, 20% or 10%. Most preferably the relative standard deviation is less than 5%. A person skilled in the art knows how to determine the average size of nanoparticles and the respective relative standard deviation.

In another preferred embodiment, the metal nanoparticle comprises only one nanoparticle. This embodiment allows for a particularly rigid quantification. Preferably the size of said one nanoparticle (e.g., NP1 or NP2) ranges from about 1 nm to about 200 nm. More preferably the size of said one nanoparticle ranges about 5 nm to about 120 nm, and even more preferably about 10 nm to about 100 nm. Most preferably, the size of said one nanoparticle ranges about 50 nm to about 80 nm. Methods for the preparation of such metal nanoparticles are known in the art and are described, for example, in Aroca, Surface-enhanced Vibrational Spectroscopy, Wiley, 2006.

It is particularly preferred that both NP1 and NP2 have a particle size of at least about 50 nm (e.g., about 50 nm to about 200 nm, particularly about 50 nm to about 100 nm). It is further preferred that NP1 and NP2 have essentially the same particle size, more preferably the same particle size.

The particle size, including the diameter (particle size in the case of spherical nanoparticles), can be determined, e.g., using 2D projection images (TEM). The longest and shortest Feret's lengths can bed averaged to determine NP's diameter (which can be measured, e.g., with the “Image J” program).

Coinage metals such as silver (Ag), gold (Au), or copper (Cu) or alloys thereof are known for their large SERS enhancement. Thus, in a preferred embodiment the metal nanoparticle comprises a metal selected from Ag, Au and Cu or alloys thereof. Generally, the metal nanoparticle employed herein may comprise any metal, alloys thereof and/or any other material which exhibits a (large) SERS enhancement. For example, Na, K, Cr, Al, U, alloys thereof and alloys thereof with any of the above coinage metals may be used. Further, it is preferred that the plasmon resonance of the metal nanoparticle occurs between 300 nm and 1500 nm. In particular, the visible (400 nm to 750 nm) to near-infrared (750 nm to 1 μm) spectral region is preferred. The region 620 nm to 1500 nm is most preferred. Here, autofluorescence of biological specimen, which decreases the image/signal contrast, can be minimized. Also, tissue is relatively transparent in this spectral region (“biologcal window”, for example, for in vivo applications).

Single particles may be spherical or non-spherical. Examples for spherical particles are solid spheres, core-shell particles and hollow spheres. Hollow nanoparticles are also referred to as nanoshells. Nanoshells can be preferable in terms of SERS sensitivity as compared to solid spheres. Further, nanoshells may be preferable when laser excitation in the red to near-Infrared (NIR) spectral region is employed. Non-spherical particles may be, inter alia, rods/ellipsoids, toroids, triangles, cubes, stars and fractal geometries. The use of said non-spherical particles may be preferred over spherical particles since non-spherical geometries lead to large electromagnetic field enhancements because of the high curvature radius. Thus, non-spherical particles can achieve particularly high sensitivity. Spherical particles provide the advantage of a high symmetry, i.e. all molecules in the SAM experience can experience the same enhancement, i.e. the same increased local electromagnetic field. Thus, spherical particles can be preferred when the application at hand focuses on a rigid quantification.

Moreover, the particles may be composite particles formed from combinations of different materials including a metal. Examples thereof are particles of the core-shell type wherein a metal shell, preferably a shell of Ag, Au or Cu, is present on a non-metallic core, e.g. a core of a metal oxide or a non-metal oxide, such as alumina, titanium dioxide or silica.

It is preferred that the first metal nanoparticle NP1 is a coinage metal particle (wherein the coinage metal may be, e.g., gold, silver, copper, or an alloy thereof), more preferably a noble metal nanoparticle, even more preferably a gold nanoparticle or a silver nanoparticle, and yet even more preferably a gold nanoparticle.

Moreover, while the first metal nanoparticle NP1 may have any shape, as explained above, it is preferred that the first metal nanoparticle is a spherical or a cubic nanoparticle, more preferably a spherical nanoparticle. It is particularly preferred that the first metal nanoparticle is a spherical nanoparticle, wherein (i) at least about 90 mol-% of the first metal nanoparticle has a roundness value of at least about 0.94, and/or (i) the relative standard deviation in the particle size distribution of the first metal nanoparticle is smaller than about 6.0%.

The roundness (or roundness value) is a parameter that is well-known in the art and is defined as follows:

$$R = \frac{4 A}{π {D}^{2}} ( D is the maximum {Feret}^{′} s diameter )$$

The roundness of a nanoparticle can be determined, e.g., as described in ACS Nano, 2013, 7, 11064. In this publication, the relevant parameter is referred to as “circularity” or “c” even though the technically correct term is roundness, as it is used herein and defined above.

The term “self-assembled monolayer” (also referred to as “SAM”) is known in the art (cf. for example Krilegisch (2005) Top Curr Chem 258, 257; Love, Chemical Reviews (2005) 105, 1103; Daniel, Chemical Reviews (2004) 104, 293; Li, Journal of Materials Chemistry (2004) 14, 2954; Welsbecker, Langmulr (1996) 12, 3763). Herein, the term “self-assembled monolayer” (SAM) is used to denote a layer which forms spontaneously when the metal nanoparticle or metal surface and compounds forming the SAM are mixed under suitable conditions. SAMs typically provide a single layer of molecules on the surface of substrates, such as metal particles. They can often be prepared simply by adding a solution of the desired molecule onto the substrate and washing off the excess. The formation of SAMs has been previously described. For example, Kriegisch (2005) Top Curr Chem 258, 257 describes the spontaneous formation of a SAM of alkyl or aryl thioles and disulfides (as precursors) on gold (and other metal) surfaces. SAMs can provide a uniform coverage of the complete surface of the metal particle. A uniform coverage of the metal nanoparticle may be advantageous with respect to quantification of Raman intensities. Quantification may, for example, be achieved by spectrally resolved detection and direct labeling (in the case of proteins: labelling of the primary antibody) in combination with reference experiments (for example, using known target molecule concentrations in immunoassays). The similar or even same molecular orientation of molecules within the SAM is very advantageous for multiplexed applications, because only selected Raman bands are observed in the spectrum (SERS selection rules, see for example Creighton in: Clark, Hester (Eds.) Advances in spectroscopy: spectroscopy of surfaces, Vol. 6, pp. 37, Wiley, 1988; Smith, Modern Raman Spectroscopy, Wiley, 2005) and an unwanted overlap of spectral contributions by a distinct moiety comprised in the SERS marker is minimized. Because the Raman intensity is proportional to the number of molecules, the formation of a SAM is also advantageous in terms of the detection limit (high sensitivity): a SAM has a large number of Raman-active groups comprised in the SERS marker per unit surface area. In addition, complete coverage of the metal nanoparticle by a SAM inhibits a direct adsorption of (bio)molecules to the particle surface.

As explained above, the term “self-assembled monolayer” as used herein typically denotes a layer formed by molecules which assemble in the form of a monolayer on a metal particle and adhere to its surface, generally due to adsorption phenomena. The expression “self-assembled monolayer of a compound X” indicates that the respective self-assembled monolayer is formed from the compound X. In such a case, the self-assembled monolayer may be formed solely from the respective compound X, or it may alternatively be formed from the compound X and one or more further compounds.

As the negatively charged substrate, with which the first metal nanoparticle NP1, having a bilayer of a long-chained cationic quaternary ammonium compound bound to its surface, is to be contacted/reacted in step (1) of the method according to the first aspect of the invention, any kind of negatively charged substrate can in principle be used. Examples of the negatively charged substrate include, in particular, a glass substrate (e.g., a glass slide), a silicon substrate (e.g., a silicon wafer), a silica substrate (e.g., a silica particle), or an indium tin oxide (ITO) substrate (e.g., an ITO plate). It is preferred that the negatively charged substrate is a glass substrate.

In step (ii) of the method according to the first aspect of the invention, the first metal nanoparticle (NP1), which is bound to the surface of the negatively charged substrate via the bilayer of the long-chained cationic quaternary ammonium compound, is subjected to an alkali metal or alkaline earth metal halide and a polar organic solvent to remove the bilayer of the long-chained cationic quaternary ammonium compound from those parts of the surface of NP1 that are not bound to the surface of the negatively charged substrate.

As the alkali metal or alkaline earth metal halide to be used in step (i) of the method according to the first aspect of the invention, in principle any alkali metal halide/halogenide and/or any alkaline earth metal halide/halogenide can be used. Corresponding examples of such alkali metal halides or alkaline earth metal halides (also referred to herein as alkali metal halogen salts or alkaline earth metal halogen salts) include, in particular, sodium fluoride, sodium chloride, sodium bromide, sodium iodide, potassium fluoride, potassium chloride, potassium bromide, potassium iodide, calcium fluoride, calcium chloride, calcium bromide, calcium iodide, magnesium fluoride, magnesium chloride, magnesium bromide, or magnesium iodide. It is preferred that the alkali metal or alkaline earth metal halide is an alkali metal halide, more preferably sodium chloride (NaCl), sodium bromide (NaBr), potassium chloride (KCl) or potassium bromide (KBr), even more preferably sodium chloride or sodium bromide, and most preferably sodium bromide. In accordance with the present invention, it is also possible to use a hydrohalogenic acid in place of the alkali metal or alkaline earth metal halide. However, as explained above, it is preferred to use an alkali metal or alkaline earth metal halide, and it is particularly preferred to use sodium bromide or sodium chloride (particularly sodium bromide).

The polar organic solvent to be used in step (ii) of the method according to the first aspect of the invention is not particularly limited, and in principle any polar organic solvent may be used. In particular, the polar organic solvent may be selected, e.g., from an alcohol (e.g. methanol, ethanol, or isopropanol), dimethyformamide (DMF), dimethyl sulfoxide (DMSO), acetone, acetonitrile (MeCN), and a mixture of any of the aforementioned polar organic solvents with water. Accordingly, the polar organic solvent to be used in step (ii) may also be a mixture of a polar organic solvent with water (e.g., a mixture of a polar organic solvent with up to 2, 3, 5, 10, 20, 30, 40 or 50 vol-% water in the final mixture). A person skilled in the art can readily choose the minimum content of the polar organic solvent in such mixtures of a polar organic solvent with water depending on the solubility of the reagents to be employed in the corresponding mixture, i.e., to ensure solubility in the respective mixture. Preferably, the polar organic solvent is an alcohol (e.g., a C₁ alkanol, particularly ethanol) or acetonitrile, more preferably it is ethanol or acetonitrile, and even more preferably the polar organic solvent is ethanol. It is furthermore preferred that the polar organic solvent (including any of the aforementioned specific or preferred examples of the polar organic solvent) is used without water or at most as a mixture with up to 20 vol-% water (more preferably up to 10 vol-% water, even more preferably up to 5 vol-% water, and still more preferably up to 2 vol-% water), and it is even more preferred that it is used without or essentially without water (e.g., without about 0.4 vol-% water).

In step (ii) of the method according to the first aspect of the invention, a mixture of an alkali metal or alkaline earth metal halide and a polar organic solvent, particularly a solution of an alkali metal or alkaline earth metal halide in a polar organic solvent (e.g., a solution of sodium bromide in ethanol), can be employed.

As a result of step (ii) of the method according to the first aspect of the invention—i.e., of subjecting the first metal nanoparticle (NP1), which is bound to the surface of the negatively charged substrate via the bilayer of the long-chained cationic quaternary ammonium compound, to an alkali metal or alkaline earth metal halide and a polar organic solvent—the bilayer of the long-chained cationic quaternary ammonium compound is removed from those parts of the surface of NP1 that are not bound to the surface of the negatively charged substrate. Accordingly, only those parts of the bilayer of the long-chained cationic quaternary ammonium compound are removed that do not bind to both the surface of NP1 and the surface of the negatively charged substrate. This step is preferably conducted such as to remove the bilayer of the long-chained cationic quaternary ammonium compound from essentially all (or, most preferably, from all) those parts of the surface of NP1 at which the bilayer of the long-chained cationic quaternary ammonium compound does not form a linkage to the surface of the negatively charged substrate.

In step (iii) of the method according to the first aspect of the invention, the first metal nanoparticle (NP1), which is bound to the surface of the negatively charged substrate via the bilayer of the long-chained cationic quaternary ammonium compound, is subjected to a compound HS—R—X (wherein R is an organic group and X is a functional group containing a sulfur atom or a nitrogen atom) and a polar organic solvent to allow the formation of a self-assembled monolayer of the compound HS—R—X on those parts of the surface of NP1 that are not bound to the negatively charged substrate.

The compound HS—R—X may be any compound comprising a thiol group (—SH) and a functional group X containing a sulfur atom or a nitrogen atom, wherein the thiol group and the group X are bound to an organic group R. The group X is preferably selected from —SH, —S(C₁₋₅ alkyl), —S-acetyl, —S—C(O)C₁₋₅ alkyl), —SCN, —NH₂, —NH(C₁₋₆ alkyl), —N(C₁₋₅ alkyl)(C₁₋₅ alkyl), —NH— acetyl, —N(C₁₋₅ alkyl)-acetyl, —NCS, and a heteroaryl containing at least one nitrogen ring atom. The group X may also be —N⁺(C₁₋₅ alkyl)₃. Moreover, it is also possible to use any other surface-seeking group in place of the group X (such as, e.g., any of the corresponding groups disclosed in U.S. Pat. No. 8,854,617 which is incorporated herein in its entirety). If X is —SH, the compound HS—R—X is a dithiol. Accordingly, a preferred example of the compound HS—R—X is a dithiol. The dithiol may be, for example, an alkanedithiol which is preferably a compound HS—(C₂₋₂₀ alkylene)-SH, more preferably a compound HS—(C₂₋₁₆ alkylene)-SH, even more preferably a compound HS—(C₄₋₁₄ alkylene)-SH, even more preferably a compound HS—(C₆₋₁₁ alkylene)-SH, or yet even more preferably 1,6-hexanedithol, 1,8-octanedithiol or 1,10-decanedithol. It is furthermore preferred that the alkylene moiety comprised in any of the aforementioned groups is linear. Accordingly, it is particularly preferred that the dithiol (or the compound HS—R—X) is a compound HS—(CH₂)₂₋₂₀—SH, more preferably a compound HS—(CH₂)₂₋₁₆—SH, even more preferably a compound HS—(CH₂)₄₋₁₄—SH, even more preferably a compound HS—(CH₂)₆₋₁₁—SH, or yet even more preferably 1,6-hexanedithiol, 1,8-octanedithiol or 1,10-decanedithiol.

The group R in the compound HS—R—X may, in principle, be any organic group. This group R can be suitably chosen to control the functionality or the SERS-activity of the resulting assembly structure, if desired. It is preferred that the group R comprises (or consists of) a SERS-active group or a Raman-active group, particularly a SERS-active group. Corresponding groups are known in the art and are described, e.g., in U.S. Pat. No. 8,854,617. For example, if the R group is benzene whose the polarizability is big, the assembly or marker to be prepared will be SERS-active.

In particular, R may be a hydrocarbyl, wherein said hydrocarbyl is optionally substituted with one or more (e.g., one, two, three or four) groups R¹, and further wherein one or more (e.g., one, two or three) —CH₂— units comprised in said hydrocarbyl are each optionally replaced by a group —R²—. Said hydrocarbyl is preferably selected from alkyl, alkenyl, alkynyl, cycloalkyl, cycloalkenyl, and aryl, particularly from C₁₋₂ alkyl, C₂₋₂₀ alkenyl, C₂₋₂₀ alkynyl, C₆₋₂₂ cycloalkyl, C₆₋₂₂ cycloalkenyl, and C₆₋₂₂ aryl.

Each R¹ is independently selected from C₁₋₅ alkyl, C₂₋₅ alkenyl, C₂₋₅ alkynyl, —(C₀₋₃ alkylene)-OH, —(C₀₋₃ alkylene)-O(C₁₋₅ alkyl), —(C₀₋₃ alkylene)-O(C₁₋₅ alkylene)-OH, —(C₀₋₃ alkylene)-O(C₁₋₅ alkylene)-O(C₁₋₅ alkyl), —(C₀₋₃ alkylene)-SH, —(C₀₋₃ alkylene)-S(C₁₋₅ alkyl), —(C₀₋₃ alkylene)-S(C₁₋₅ alkylene)-SH, —(C₀₋₃ alkylene)-S(C₁₋₅ alkylene)-S(C₁₋₅ alkyl), —(C₀₋₃ alkylene)-NH₂, —(C₀₋₃ alkylene)-NH(C₁₋₅ alkyl), —(C₀₋₃ alkylene)-N(C₁₋₅ alkyl)(C₁₋₅ alkyl), —(C₀₋₃ alkylene)-halogen, —(C₀₋₃ alkylene)-(C₁₋₅ haloalkyl), —(C₀₋₃ alkylene)-O—(C₁₋₅ haloalkyl), —(C₀₋₃ alkylene)-CF₃, —(C₀₋₂ alkylene)-CN, —(C₀₋₃ alkylene)-NO₂, —(C₀₋₃ alkylene)-Na, —(C₀₋₃ alkylene)-CHO, —(C₀₋₃ alkylene)-CO—(C₁₋₅ alkyl), —(C₀₋₃ alkylene)-COOH, —(C₀₋₃ alkylene)-CO—O—(C₁₋₅ alkyl), —(C₀₋₃ alkylene)-O—CO—(C₁₋₅ alkyl), —(C₀₋₃ alkylene)-CO—NH₂, —(C₀₋₃ alkylene)-CO—NH(C₁₋₅ alkyl), —(C₀₋₃ alkylene)-CO—N(C₁₋₅ alkyl)(C₁₋₅ alkyl), —(C₀₋₃ alkylene)-NH—CO—(C₁₋₅ alkyl), —(C₀₋₃ alkylene)-N(C₁₋₅ alkyl)-CO—(C₁₋₅ alkyl), —(C₀₋₃ alkylene)-SO₂—NH₂, —(C₀₋₃ alkylene)-SO₂—NH(C₁₋₅ alkyl), —(C₀₋₃ alkylene)-SO₂—N(C₁₋₅ alkyl), —(C₀₋₃ alkyl), —(C₀₋₃ alkylene)-NH—SO₂—(C₁₋₅ alkyl), —(C₀₋₃ alkylene)-N(C₁₋₅ alkyl)-SO₂(C₁₋₅ alkyl), —(C₀₋₃ alkylene)-carbocyclyl, and —(C₀₋₃ alkylene)-heterocyclyl. Preferably, each R¹ is independently selected from C₁₋₅ alkyl, C₂₋₅ alkenyl, C₂₋₅ alkynyl, —OH, —O(C₁₋₅ alkyl), —O(C₁₋₅ alkylene)-OH, —O(C₁₋₅ alkylene)-O(C₁₋₅ alkyl), —SH, —S(C₁₋₅ alkyl), —S(C₁₋₅ alkylene)-SH, —S(C₁₋₅ alkylene)-S(C₁₋₅ alkyl), —NH₂, —NH(C₁₋₅ alkyl), —N(C₁₋₅ alkyl)(C₁₋₅ alkyl), halogen, C₁₋₅ haloalkyl, —O—(C₁₋₅ haloalkyl), —CF₃, —CN, —NO₂, —N, —CHO, —CO—(C₁₋₅ alkyl), —COOH, —CO—O—(C₁₋₅ alkyl), —O—CO—(C₁₋₅ alkyl), —CO—NH₂, —CO—NH(C₁₋₅ alkyl), —CO—N(C₁₋₅ alkyl)(C₁₋₅ alkyl), —NH—CO—(C₁₋₅ alkyl), —N(C₁₋₅ alkyl)-CO—(C₁₋₅ alkyl), —SO₂—NH₂, —SO₂—NH(C₁₋₅ alkyl), —SO₂—N(C₁₋₅ alkyl)(C₁₋₅ alkyl), —NH—SO₂—(C₁₋₅ alkyl), and —N(C₁₋₅ alkyl)-SO₂—(C₁₋₅ alkyl). More preferably, each R₁ is independently selected from C₁₋₅ alkyl, C₂₋₅ alkenyl, C₂₋₅ alkynyl, —OH, —O(C₁₋₅ alkyl), —O(C₁₋₅ alkylene)-OH, —O(C₁₋₅ alkylene)-O(C₁₋₅ alkyl), —SH, —S(C₁₋₅ alkyl), —S(C₁₋₅ alkylene)-SH, —S(C₁₋₅ alkylene)-S(C₁₋₅ alkyl), —NH₂, —NH(C₁₋₅ alkyl), —N(C₁₋₅ alkyl)(C₁₋₅ alkyl), halogen, C₁₋₅ haloalkyl, —CF₃, and —CN, particularly from C₁₋₅ alkyl (e.g., methyl or ethyl), —OH, —O(C₁₋₄ alkyl) (e.g., —OCH₃ or —OCH₂CH₃), —NH₂, —NH(C₁₋₄ alkyl) (e.g., —NHCH₃), —N(C₁₋₄ alkyl)(C₁₋₄ alkyl) (e.g., —N(CH₃)₂), halogen (e.g., —F, —Cl, —Br, or —I), —CF₃, and —CN.

Each R² is independently selected from —O—, —CO—, —C(═O)—, —O—C(═O), —N(R^{2a}), —N(R^{2a})—CO—, —CO—N(R²)—, —N(R^{2a})—CO—N(R^{2a})—, —N(R^{2a})—(═O)O—, —O—C(═O)—N(R^{2a})—, —N(R^{2a})—C(NH₂)N—, —N═C(NH₂)—N(R₂ₐ)—, —N(R^{2a})—C(═N—CN)—N(R^{2a})—, —N(R^{2a})—C(═N—R^{2a})—N(R^{2a})—, —N(R^{2a})—C(═N—R^{2a}), —C(═N—R^{2a})—N(R^{2a})—, —N(R₂ₐ)—C(═CH—NO₂)—N(R₂ₐ)—, —N(R^{2a})—C(═N—NO₂)—N(R^{2a})—, —N(R^{2a})—C(═N—CN)—, —C(═N—CN)—N(R^{2a})—, —N(R^{2a})—C(═CH—NO₂)—, —C(═CH—NO₂)N(R^{2a})—, —N(R^{2a})—C(═N—NO₂)—, —C(═N—NO₂)—N(R^{2a})—, —S—, —SO—, —SO₂—, —SO₂—N(R^{2a})—, —N(R^{2a})—SO₂—, —N(R^{2a})—SO₂—N(R²)—, —SO—N(R^{2a})—, —N(R^{2a})—SO—, —N(R^{2a})—SO—N(R^{2a})—, —C(═S)O—, —O—C(═S)—, —C(═O)S—, —S—C(═O)—, —N(R^{2a})—C(═S)—, —C(═S)—N(R^{2a})—, —N(R^{2a})—C(═S)—N(R^{2a})—, —N(R^{2a})—C(═S)—O—, —O—C(═S)—N(R₂ₐ)—, —N(R^{2a})—C(═O)—S—, —S—C(═O)—N(R^{2a}), —S—C(═N—R^{2a})—N(R^{2a}), —N(R^{2a})C(═N—R^{2a})—S—, —S—C(═N—CN)N(R^{2a}), —N(R^{2a})C(═N—CN)—S—, —S—C(═N—NO₂)—N(R^{2a})—, —N(R^{2a})—C(═N—NO₂)—S—, —O—C(═N—R^{2a})—N(R^{2a})—, —N(R^{2a})—C(═N—R^{2a})—O—, —O—C(═N—CN)—N(R^{2a}), —N(R^{2a})—C(═N—CN)—O—, —O—C(═N—NO₂)—N(R^{2a})—, and —N(R₂ₐ)—C(═N—NO₂)—O—, wherein each R^{2a} is independently selected from hydrogen and C₁₋₅ alkyl (e.g., methyl or ethyl). Preferably, each R² is independently selected from —O—, —CO—, —C(═O)O—, —O—C(O)—, —N(R^{2a})—, —N(R^{2a})—CO—, —CO—N(R^{2a})—, —S—, —SO—, —SO₂—, —SO₂—N(R^{2a}), and —N(R^{2a})—SO₂—, wherein each R² is independently selected from hydrogen and C₁₋₅ alkyl.

Moreover, R may also be a hydrocarbyl, wherein said hydrocarbyl is optionally substituted with one or more (e.g., one, two, three or four) groups R¹ (as defined above), and further wherein one or more (e.g., one, two, three, four, five, or six) carbon atoms comprised in said hydrocarbyl are each optionally replaced by a heteroatom independently selected from oxygen, sulfur and nitrogen.

If the compound HS—R—X is a SERS-active compound HS—R—X, then it is preferred that R comprises one or more aryl groups, one or more heteroaryl groups, one or more double bonds (e.g., two or more carbon-to-carbon double bonds, particularly two or more conjugated carbon-to-carbon double bonds), and/or one or more triple bonds (e.g., two or more carbon-to-carbon triple bonds, particularly two or more conjugated carbon-to-carbon triple bonds). Likewise, if the compound HS—R—X s a Raman-active compound HS—R—X, then it is preferred that R comprises one or more aryl groups, one or more heteroaryl groups, one or more double bonds (e.g., two or more carbon-to-carbon double bonds, particularly two or more conjugated carbon-to-carbon double bonds), and/or one or more triple bonds (e.g., two or more carbon-to-carbon triple bonds, particularly two or more conjugated carbon-to-carbon triple bonds). Thus, the group R in the compound HS—R—X may be, for example, an arene (e.g., benzene), a heteroarene, or polyene or a polyyne. Accordingly, the compound HS—R—X may be, e.g., a SERS-active (or Raman-active) dithiol (e.g., a polyene dithiol), particularly a compound HS—R—SH, wherein R is a polyene (such as, e.g., a group —CH═CH—CH═CH—, —CH═CH—CH═CH—CH═CH— or —CH═CH—CH═CH—CH═CH—CH═CH—), a polyyne, an arene (such as, e.g., a group —C₆H₄—), a heteroarene, or a combination of two or more of the aforementioned groups (such as, e.g., an arene-polyene-arene). For instance, the compound HS—R—X may be a compound HS—C₆H₄—CH═CH—CH═CH—C₆H₄—SH.

For assembly, the compound HS—R—X may also be, e.g., a compound HS—(C₁₁ alkylene)-N(CH₃)₃ (MUTAB), or a compound HS—(C₈ alkyl)-SH (octanedithiol), or a compound comprising both a thiol terminus and a cationic quaternary ammonium terminus.

In step (iii) of the method according to the first aspect of the invention, a self-assembled monolayer of the compound HS—R—X is formed on those parts of the surface of NP1 that are not bound to the negatively charged substrate, i.e., on those parts of the surface of NP1 from which the bilayer of the long-chained cationic quaternary ammonium compound was removed in step (i). The compound HS—R—X can bind to the surface of NP1 via its thiol group (—SH) and can thereby form the self-assembled monolayer on the surface of NP1.

It is possible to employ only a single type/species of the compound HS—R—X in step (iii) of the method according to the first aspect of the invention, in which case the self-assembled monolayer of the compound HS—R—X that is formed on the surface of NP1 composed only of this single type of compound HS—R—X. Alternatively, it is also possible to use two or more different compounds HS—R—X in step (iii), i.e. two or more compounds HS—R—X that are structurally different from one another, in which case the resulting self-assembled monolayer that is formed on the surface of NP1 will be composed of these two or more different compounds HS—R—X.

Moreover, it is possible to use a single type of the compound HS—R—X or two or more different compounds HS—R—X in step (ill) of the method according to the first aspect of the invention, as described above, without employing any other thiol-containing compounds in this step. However, it is also possible to use one or more further compounds HS—R, wherein R is as defined herein above, in addition to the compound HS—R—X in step (ii) of the method according to the first aspect of the invention. In this case, a self-assembled monolayer will be formed from the compound HS—R—X and from the one or more compounds HS—R on the surface of NP1. For example, a compound HS—(C₈ alkyl)-SH (which is an example of the compound HS—R—X and serves as a linker for attaching the second metal nanoparticle NP2) and a compound HS-benzene (i.e., thiophenol, which is an example of the compound HS—R and serves as a SERS-active reporter) can be employed together in step (iii), e.g., in a molar ratio of 1:1, in order to prepare SERS-active dimeric nanoparticles.

Thus, a mixed self-assembled monolayer of an alkyl dithiol and a Raman-active thiol molecule can be prepared, e.g., by following the approach as described above. As also illustrated in the appended examples, dimers linked by 1,8-octane dithiol (C8) with different amounts of thiophenol (TP) as Raman-active molecule can thus be prepared. These dimers can be prepared with different ratios of C8 and TP (e.g., 99:1, 9:1, 3:1, 1:1, 1:3, 1:9, or 1:99). As demonstrated in the examples, in all such cases dimers are formed, but only dimers with a ratio of 3:1 C8/TP or higher were SERS-active (measured at the ensemble level).

Moreover, also other Raman reporter molecules such as 4-nitrothiophenol (NTP), 7-mercapto-4-methylcoumarin (MMC), thio-2-naphthol (TN), 2,3,5,6-tetrafluoro-4-mercaptobenzoic acid (TFMBA), mercapto-4-methyl-5-thioacetic acid (MMTA), 2-bromo-4-mercaptobenzoic acid (BMBA), ethyl(2E,4E,6E,8E,10E,12E,14E)-15-(4-(tert-butylthio)phenyl)pentadeca-2,4,6,8,10,12,14-heptanoate (Polyene 7DB), or ethyl(2E,4E)-5-(4-(tert-butylthio)phenyl)penta-2,4-dienoate (Polyene 2DB) can be used in place of thiophenol to build a mixed monolayer.

In particular, the following protocol can be used in step (I) of the method according to the first aspect of the invention, i.e. the SAM formation step, to obtain dimers with a dual SAM of TP and C8:

Linker dual SAM formation of C and TP (50:50) on the 1^{st} AuNS in EtOH containing NaBr
1 mM octanedithiol (C8) in EtOH 2.5 mL and 1 mM thiophenol (TP) in EtOH 2.5 mL was prepared and mixed together.
254 mM NaBr in H₂O 20 μL was added to the mixed C8/TP solution in order to adjust 1 mM NaBr.
A glass slide was cleaned by water and EtOH.
It was immersed in the linker solution for 1.5 h at 30° C.

The polar organic solvent to be used in step (IN) of the method according to the first aspect of the invention is not particularly limited, and in principle any polar organic solvent may be used. In particular, the same polar organic solvents as described above in connection with step (H) can be used. Thus, the polar organic solvent may be selected, e.g., from an alcohol (e.g. methanol, ethanol, or isopropanol), dimethylformamide (DMF), dimethyl sulfoxide (DMSO), acetone, acetonitrile (MeCN), and a mixture of any of the aforementioned polar organic solvents with water. Accordingly, the polar organic solvent to be used in step (IN) may also be a mixture of a polar organic solvent with water (e.g., a mixture of a polar organic solvent with up to 2, 3, 5, 10, 20, 30, 40 or 50 vol-% water in the final mixture). A person skilled in the art can readily choose the minimum content of the polar organic solvent in such mixtures of a polar organic solvent with water depending on the solubility of the reagents to be employed in the corresponding mixture, i.e., to ensure solubility in the respective mixture. Preferably, the polar organic solvent is an alcohol (e.g., a C₁₋₅ alkanol, particularly ethanol) or acetonitrile, more preferably it is ethanol or acetonitrile, and even more preferably the polar organic solvent is ethanol. It is furthermore preferred that the polar organic solvent (including any of the aforementioned specific or preferred examples of the polar organic solvent) is used without water or at most as a mixture with up to 20 vol-% water (more preferably up to 10 vol-% water, even more preferably up to 5 vol-% water, and still more preferably up to 2 vol-% water), and it is even more preferred that it is used without or essentially without water (e.g., without about 0.4 vol-% water). In step (iii), a mixture of the compound HS—R—X and a polar organic solvent, particularly a solution of the compound HS—R—X in a polar organic solvent (e.g., a solution of a dithiol in ethanol), can be employed.

While it is possible to use the same or different polar organic solvents in step (ii) and step (iii) of the method according to the first aspect of the invention, it is preferred to use the same polar organic solvent in both of these steps, which also allows to simultaneously conduct steps (ii) and (iii). In particular, steps (i) and (iii) can be conducted simultaneously by subjecting NP1, which is bound to the surface of the negatively charged substrate via the bilayer of the long-chained cationic quaternary ammonium compound, to an alkali metal or alkaline earth metal halide, a compound HS—R—X and a polar organic solvent to remove the bilayer of the long-chained cationic quaternary ammonium compound from those parts of the surface of NP1 that are not bound to the surface of the negatively charged substrate and to allow the formation of a self-assembled monolayer of the compound HS—R—X on those parts of the surface of NP1. If steps (ii) and (iii) are carried out simultaneously as described above, a mixture of the alkali metal or alkaline earth metal halide, the compound HS—R—X and the polar organic solvent, particularly a solution of the alkali metal or alkaline earth metal halide and the compound HS—R—X in the polar organic solvent (e.g., a solution of sodium bromide and a dithiol compound in ethanol), can be employed.

In step (iv) of the method according to the first aspect of the invention, NP1 which is bound to the surface of the negatively charged substrate via the bilayer of the long-chained cationic quaternary ammonium compound and which has a self-assembled monolayer of the compound HS—R—X bound to those parts of its surface that are not bound to the negatively charged substrate, is contacted with a polar organic solvent, an alkali metal or alkaline earth metal halide and a second metal nanoparticle (NP2), wherein NP2 has a bilayer of a long-chained cationic quaternary ammonium compound bound to its surface, to obtain a conjugate of NP1 and NP2, wherein NP1 and NP2 are linked together in said conjugate via a part of the self-assembled monolayer of the compound HS—R—X, said part being bound to the metal surface of both NP1 and NP2, wherein said conjugate of NP1 and NP2 is bound to the surface of the negatively charged substrate via the bilayer of the long-chained cationic quaternary ammonium compound that is bound to the surface of NP1.

The polar organic solvent to be used in step (iv) of the method according to the first aspect of the invention is not particularly limited, and in principle any polar organic solvent may be used, including any of the polar organic solvents described herein above in connection with step (ii) or (iii). Thus, the polar organic solvent may be selected, e.g., from an alcohol (e.g. methanol, ethanol, or isopropanol), dimethylformamide (DMF), dimethyl sulfoxide (DMSO), acetone, acetonitrile (MeCN), and a mixture of any of the aforementioned polar organic solvents with water. Accordingly, the polar organic solvent to be used in step (iv) of the method according to the first aspect of the invention may also be a mixture of a polar organic solvent with water (e.g., a mixture of a polar organic solvent with up to 2, 3, 5, 10, 20, 30, 40 or 50 vol-% water in the final mixture). A person skilled in the art can readily choose the minimum content of the polar organic solvent in such mixtures of a polar organic solvent with water depending on the solubility of the reagents to be employed in the corresponding mixture, i.e., to ensure solubility in the respective mixture. Preferably, the polar organic solvent is an alcohol (e.g., a C₁₋₅ alkanol, particularly ethanol) or acetonitrile, more preferably it is ethanol or acetonitrile, and even more preferably the polar organic solvent to be used in step (iv) is acetonitrile. It is furthermore preferred that the polar organic solvent (including any of the aforementioned specific or preferred examples of the polar organic solvent) is used without water or at most as a mixture with up to 20 vol-% water (more preferably up to 10 vol-% water, even more preferably up to 5 vol-% water, and still more preferably up to 2 vol-% water), and it is even more preferred that it is used without or essentially without water (e.g., without about 0.4 vol-% water). In particular, in step (iv), a mixture of an alkali metal or alkaline earth metal halide, a second metal nanoparticle (NP2) and a polar organic solvent (e.g., a mixture of sodium bromide and NP2 in acetonitrile) can be employed.

As the alkali metal or alkaline earth metal halide to be used in step (iv) of the method according to the first aspect of the invention, in principle any alkali metal halide/halogenide and/or any alkaline earth metal halide/halogenid can be used, including those described herein above in connection with step (i). Thus, corresponding examples of such alkali metal halides or alkaline earth metal halides (also referred to herein as alkali metal halogen salts or alkaline earth metal halogen salts) include, in particular, sedum fluoride, sodium chloride, sodium bromide, sodium iodide, potassium fluoride, potassium chloride, potassium bromide, potassium iodide, calcium fluoride, calcium chloride, calcium bromide, calcium iodide, magnesium fluoride, magnesium chloride, magnesium bromide, or magnesium iodide. It is preferred that the alkali metal or alkaline earth metal halide to be used in step (iv) is an alkali metal halide, more preferably sodium chloride (NaCl), sodium bromide (NaBr), potassium chloride (KCl) or potassium bromide (KBr), even more preferably sodium chloride or sodium bromide, and most preferably sodium bromide. In accordance with the present invention, it is also possible to use a hydrohalogenic acid in place of the alkali metal or alkaline earth metal halide. However, as explained above, it is preferred to use an alkali metal or alkaline earth metal halide in step (iv) of the method according to the first aspect of the invention, and it is particularly preferred to use sodium bromide or sodium chloride (particularly sodium bromide).

The second metal nanoparticle (NP2) to be used in step (v) of the method according to the first aspect of the invention is as described above. It is particularly preferred that the second metal nanoparticle NP2 is a coinage metal particle (wherein the coinage metal may be, e.g., gold, silver, copper, or an alloy thereof), more preferably a noble metal nanoparticle, even more preferably a gold nanoparticle or a silver nanoparticle, and yet even more preferably a gold nanoparticle. NP1 and NP2 may be made of the same material (e.g., they may both be gold nanoparticles) or they may be made of different materials.

Moreover, while the second metal nanoparticle NP2 may have any shape, it is preferred that the second metal nanoparticle is a spherical or a cubic nanoparticle, more preferably a spherical nanoparticle. It is particularly preferred that the second metal nanoparticle is a spherical nanoparticle, wherein (i) at least about 90 mol-% of the second metal nanoparticle has a roundness value of at least about 0.94, and/or (ii) the relative standard deviation in the particle size distribution of the second metal nanoparticle is smaller than about 6.0%.

The second metal nanoparticle (NP2) can be subjected to chemical etching before it is used in step (iv). As explained above, a chemical etching step is advantageous as its allows to obtain very round and homogenous particles although it is not necessary to conduct chemical etching (thus, round nanoparticles prepared without etching, e.g., in accordance with Part. Part. Syst Charact. 2014, 31, 266-273 can also be used). The chemical etching can be conducted, e.g., as described in Ruan et al., 2014 or as described in the examples.

The second metal nanoparticle NP2 which is employed in step (Iv) of the method according to the first aspect of the invention has a bilayer of a long-chained cationic quaternary ammonium compound bound to its surface. The long-chained cationic quaternary ammonium compound is as described and defined herein above in connection with step (1) of the method according to the first aspect of the invention. While it is possible that the long-chained cationic quaternary ammonium compound forming a bilayer on the surface of the first metal nanoparticle NP1 to be used in step (i) is different from the long-chained cationic quaternary ammonium compound forming a bilayer on the surface of the second metal nanoparticle NP2 to be used in step (iv), it is preferred that the same long-chained cationic quaternary ammonium compound is bound to the surface of both NP1 (to be used in step (i)) and NP2 (to be used in step (iv)).

In step (v) of the method according to the first aspect of the invention, the conjugate of NP1 and NP2, which is bound to the surface of the negatively charged substrate via the bilayer of the long-chained cationic quaternary ammonium compound that is bound to the surface of NP1, and which has a bilayer of the long-chained cationic quaternary ammonium compound bound to the surface of NP2, is subjected to a compound containing an N,N,N-trialkylammonium group and/or a thiol group, an alkali metal or alkaline earth metal halide and a polar organic solvent to remove the bilayer of the respective long-chained cationic quaternary ammonium compound from both NP1 and NP2, to allow the formation of a self-assembled monolayer of the compound containing an N,N,N-trialkylammonium group and/or a thiol group on those parts of the surface of both NP1 and NP2 that are not bound by the self-assembled monolayer of the compound HS—R—X, and to release the conjugate of NP1 and NP2 from the surface of the negatively charged substrate to provide the dimeric nanoparticle assembly. The dimeric nanoparticle assembly thus obtained comprises NP1 and NP2, wherein NP1 comprised in the dimeric nanoparticle assembly has a self-assembled monolayer of the compound containing an N,N,N-trialkylammonium group and/or a thiol group bound to one part of its surface and a self-assembled monolayer of the compound HS—R—X bound to the remaining part of its surface, wherein NP1 and NP2 are linked together via a part of the self-assembled monolayer of the compound HS—R—X, which part is bound to the surface of both NP1 and NP2, and wherein NP2 comprised in the dimeric nanoparticle assembly has a self-assembled monolayer of the compound containing an N,N,N-trialkylammonium group and/or a thiol group bound to the part of its surface that is not bound by the self-assembled monolayer of the compound HS—R—X.

In step (v) of the method according to the first aspect of the invention, the compound containing an N,N,N-trialkylammonium group and/or a thiol group can, in principle, be any compound containing an N,N,N-trialkylammonium group, any compound containing a thiol group (—SH), or any compound containing both an N,N,N-trialkylammonium group and a thiol group. In particular, it is advantageous to use any such compound with a surface seeking group which can electrostatically or sterically stabilize the dimer after desorption. For example, the compound containing an N,N,N-trialkylammonium group and/or a thiol group may be a PEG thiol compound or 11-mercaptoundecanoic acid (MUA). The compound containing an N,N,N-trialkylammonium group and/or a thiol group may also be a hydrocarbyl-SH or a hydrocarbyl-N⁺(C₁₋₅ alkyl)₃, wherein the hydrocarbyl comprised in said hydrocarbyl-SH or in said hydrocarbyl-N⁺(C₁₋₅ alkyl) is optionally substituted with one or more (e.g., one, two or three) groups independently selected from selected from —SH and —N⁺(C₁₋₅ alkyl)₃.

Preferably, the compound containing an N,N,N-trialkylammonium group and/or a thiol group is a compound containing both an N,N,N-trialkylammonium group and a thiol group (which is also referred to herein as an N,N,N-trialkylammonium-substituted thiol compound), more preferably it is an N,N,N-tri(C₁₋₄ alkyl)ammonium-alkanethiol, even more preferably a compound N⁺(C₁₋₄ alkyl)₃-(C₂₋₁₆ alkylene)-SH, even more preferably a compound N⁺(CH₃)₃—(C₂₋₁₆ alkylene)-SH, still more preferably a compound N⁺(CH₃)₃—(CH₂)₂₋₁₆—SH, and most preferably a compound N⁺(CH₃)₃—(CH₂)₁₁—SH.

If the compound containing an N,N,N-trialkylammonium group and/or a thiol group comprises an N,N,N-trialkylammonium group, it can be employed in the form of a salt of the respective compound, e.g., a halide salt (such as a chloride or a bromide). For example, if the compound is N⁺(CH₃)₃—CH₂)₁₁—SH, then the corresponding bromide salt can be used in step (v) of the method according to the first aspect of the invention, which is also referred to as (11-mercaptoundecyl)-N,N,N-trimethylammonium bromide (or “MUTAB”).

As the alkali metal or alkaline earth metal halide to be used in step (v) of the method according to the first aspect of the invention, in principle any alkali metal halide/halogenide and/or any alkaline earth metal halidehalogenid can be used, including those described herein above in connection with any of the preceding steps. Thus, corresponding examples of such alkali metal halides or alkaline earth metal halides (also referred to herein as alkali metal halogen salts or alkaline earth metal halogen salts) include, in particular, sodium fluoride, sodium chloride, sodium bromide, sodium iodide, potassium fluoride, potassium chloride, potassium bromide, potassium iodide, calcium fluoride, calcium chloride, calcium bromide, calcium iodide, magnesium fluoride, magnesium chloride, magnesium bromide, or magnesium iodide. It is preferred that the alkali metal or alkaline earth metal halide to be used in step (v) is an alkali metal halide, more preferably sodium chloride (NaCl), sodium bromide (NaBr), potassium chloride (KCl) or potassium bromide (KBr), even more preferably sodium chloride or sodium bromide, and most preferably sodium bromide. In accordance with the present invention, it is also possible to use a hydrohalogenic acid in place of the alkali metal or alkaline earth metal halide. However, as explained above, it is preferred to use an alkali metal or alkaline earth metal halide in step (v), and it is particularly preferred to use sodium bromide or sodium chloride (particularly sodium bromide).

The polar organic solvent to be used in step (v) of the method according to the first aspect of the invention is not particularly limited, and in principle any polar organic solvent may be used, including any of the polar organic solvents described herein above in connection with any of the preceding steps. Thus, the polar organic solvent may be selected, e.g., from an alcohol (e.g. methanol, ethanol, or isopropanol), dimethylformamide (DMF), dimethyl sulfoxide (DMSO), acetone, acetonitrile (MeCN), and a mixture of any of the aforementioned polar organic solvents with water. Accordingly, the polar organic solvent to be used in step (v) of the method according to the first aspect of the invention may also be a mixture of a polar organic solvent with water (e.g., a mixture of a polar organic solvent with up to 2, 3, 5, 10, 20, 30, 40 or 50 vol-% water in the final mixture). A person skilled in the art can readily choose the minimum content of the polar organic solvent in such mixtures of a polar organic solvent with water depending on the solubility of the reagents to be employed in the corresponding mixture, i.e., to ensure solubility in the respective mixture. Preferably, the polar organic solvent is an alcohol (e.g., a C₁₋₅ alkanol, particularly ethanol) or acetonitrile, more preferably it is ethanol or acetonitrile, and even more preferably the polar organic solvent to be used in step (v) of the method according to the first aspect of the invention is ethanol. It is furthermore preferred that the polar organic solvent (including any of the aforementioned specific or preferred examples of the polar organic solvent) is used without water or at most as a mixture with up to 20 vol-% water (more preferably up to 10 vol-% water, even more preferably up to 5 vol-% water, and still more preferably up to 2 vol-% water), and it is even more preferred that it is used without or essentially without water (e.g., without about 0.4 vol-% water).

The release of the conjugate of NP1 and NP2 from the surface of the negatively charged substrate (whereby the dimeric nanoparticle assembly is provided) can be facilitated or effected, e.g., by using sonication. The use of sonication in this step is particularly advantageous as it provides a simple and effective means for desorbing the conjugate/dimer of NP1 and NP2 from the negatively charged substrate. Other approaches for facilitating/effecting the release of the conjugate of NP1 and NP2 from the surface of the negatively charged substrate can be also used. For example, it is also possible to change the ionic strength of the solution in step (v) and counter the interaction between the negatively charged substrate and the point where NP1s is attached to the negatively charged substrate. After desorption from the negatively charged substrate, all free/accessible parts of the surface of the conjugate of NP1 and NP2 will be bound by the compound containing an N,N,N-trialkylammonium group and/or a thiol group.

The method according to the first aspect of the invention may further comprise a step of coupling a binding molecule to the dimeric nanoparticle assembly. The binding molecule can be coupled, e.g., to a functional group comprised in the self-assembled monolayer on either NP1 or NP2, or both, or it can be coupled to an encapsulating layer, i.e., a silica shelf/encapsulation or a polymer (e.g., natural and synthetic polymer like latex, polystyrene, and bio-material like protein, lipid, and sugar) shell/encapsulation, that can be formed on the dimeric nanoparticle assembly. Such encapsulating layers are described, e.g., in U.S. Pat. No. 8,854,617 (which is incorporated herein by reference). The binding molecule is preferably an antibody or an antigen-binding fragment thereof.

In a second aspect, the present invention relates to a dimeric nanoparticle assembly which is obtainable by (or obtained by) the method of the first aspect of the invention.

In a third aspect, the invention provides a method of preparing a core-satellite nanoparticle assembly, the method comprising:
(i) subjecting a first metal nanoparticle (NP1), having a bilayer of a long-chained cationic quaternary ammonium compound bound to its surface, to a compound containing an N,N,N-trialkylammonium group and a thiol group, an alkali metal or alkaline earth metal halide and a polar organic solvent to remove the bilayer of the long-chained cationic quaternary ammonium compound from the surface of NP1 and to allow the formation of a self-assembled monolayer of the compound containing an N,N,N-trialkylammonium group and a thiol group on the surface of NP1; and
(ii) contacting NP1, which has a self-assembled monolayer of the compound containing an N,N,N-trialkylammonium group and a thiol group on its surface, with a molar excess of negatively charged nanoparticles to obtain the core-satellite nanoparticle assembly,
   wherein the core-satellite nanoparticle assembly thus obtained comprises NP1 having a self-assembled monolayer of the compound containing an N,N,N-trialkylammonium group and a thiol group bound to its surface, and wherein the negatively charged nanoparticles are bound to the outer surface of the self-assembled monolayer of the compound containing an N,N,N-trialkylammonium group and a thiol group.

The first metal nanoparticle (NP1), the long-chained cationic quaternary ammonium compound, the compound containing an N,N,N-trialkylammonium group and a thiol group (i.e., both an N,N,N-trialkylammonium group and a thiol group), the alkali metal or alkaline earth metal halide, and the polar organic solvent to be used in step (1) of the method according to the third aspect of the invention are as described and defined herein above in connection with the method according to the first aspect of the invention.

In step (i) of the method according to the third aspect of the invention, the bilayer of the long-chained cationic quaternary ammonium compound is removed from the surface of NP1 and a self-assembled monolayer of the compound containing an N,N,N-trialkylammonium group and a thiol group is formed on the surface of NP1. In the self-assembled monolayer thus formed, the compound containing an N,N,N-trialkylammonium group and a thiol group binds to the metal surface of NP1 via its thiol group while the N,N,N-trialkylammonium group of this compound allows the attachment of the negatively charged nanoparticles (“satellites”) in step (ii), particularly via electrostatic attraction between the positively charged ammonium group and the negatively charged nanoparticles.

In step (ii) of the method according to the third aspect of the invention, NP1 is contacted/reacted with an excess of the negatively charged nanoparticles, preferably with at least a 50-fold molar excess, more preferably at least a 100-fold molar excess, even more preferably at least a 200-fold molar excess of the negatively charged nanoparticles. It is advantageous to employ a high molar excess of the negatively charged nanoparticles in order to ensure that the complete (outer) surface of the self-assembled monolayer of the compound containing an N,N,N-trialkylammonium group and a thiol group is bound (or covered) by the negatively charged nanoparticles.

The negatively charged nanoparticles may be, e.g., citrate-capped metal nanoparticles (i.e., metal nanoparticles having a monolayer of citrate molecules bound to their surface). Moreover, the negatively charged nanoparticles preferably have a smaller particle size than NP1. In particular, it is preferred that the particle size of the negatively charged nanoparticles is ⅕ or less of the particle size of NP1, more preferably 1/10 or less, even more preferably 1/50 or less, and still more preferably 1/100 or less of the particle size of NP1. The negatively charged nanoparticles are otherwise as described and defined herein above in the first aspect of the invention in connection with NP1 and NP2, particularly with respect to their material (e.g., the negatively charged nanoparticles may be gold or silver nanoparticles) and their shape (e.g., spherical).

In a fourth aspect, the present invention relates to a core-satellite nanoparticle assembly which is obtainable by (or obtained by) the method of the third aspect of the invention.

In a fifth aspect, the invention provides a method of preparing a functionalized nanoparticle, the method comprising:
(1) contacting a first metal nanoparticle (NP1), having a bilayer of a long-chained cationic quaternary ammonium compound bound to its surface, with a negatively charged substrate to obtain NP1 bound to the surface of the negatively charged substrate;
(ii) subjecting NP1, which is bound to the surface of the negatively charged substrate via the bilayer of the long-chained cationic quaternary ammonium compound, to an alkali metal or alkaline earth metal halide and a polar organic solvent to remove the bilayer of the long-chained cationic quaternary ammonium compound from those parts of the surface of NP1 that are not bound to the surface of the negatively charged substrate;
(iii) subjecting NP1, which is bound to the surface of the negatively charged substrate via the bilayer of the long-chained cationic quaternary ammonium compound, to a thiolated biomolecule and a polar organic solvent to allow the formation of a self-assembled monolayer of the thiolated biomolecule on those parts of the surface of NP1 that are not bound to the negatively charged substrate; and
(iv) subjecting NP1, which is bound to the surface of the negatively charged substrate via the bilayer of the long-chained cationic quaternary ammonium compound and which has a self-assembled monolayer of the thiolated biomolecule bound to those parts of its surface that are not bound to the negatively charged substrate, to a thiolated biomolecule, an alkali metal or alkaline earth metal halide and a polar organic solvent to remove the bilayer of the long-chained cationic quaternary ammonium compound from NP1, allow the formation of a self-assembled monolayer of the thiolated biomolecule on those parts of the surface of NP1 from which the bilayer of the long-chained cationic quaternary ammonium compound is removed, and release NP1 having a self-assembled monolayer of the respective thiolated biomolecule bound to its surface from the surface of the negatively charged substrate to provide the functionalized nanoparticle.

Steps (I) and (i) of the method according to this fifth aspect of the invention can be carried out as described herein above in connection with steps (i) and (II) of the method of the first aspect of the invention. Accordingly, the first metal nanoparticle (NP1), the long-chained cationic quaternary ammonium compound, the negatively charged substrate, the alkali metal or alkaline earth metal halide, and the polar organic solvent to be used in the method according to the fifth aspect (i.e., in any of the steps of the method according to the fifth aspect) are as described and defined herein above in connection with the method according to the first aspect of the invention.

The thiolated biomolecule which is used in step (I) of the method according to this fifth aspect can, in principle, be any biomolecule having a thiol (—SH) group, including biomolecules that naturally contain one or more thiol groups as well as biomolecules that have been modified to contain one or more thiol groups. A biomolecule of interest may also be modified by attaching a thiol group via a linker (e.g., an alkyl linker) to the biomolecule. A preferred example of the thiolated biomolecule is a thiolated nucleic acid, and more preferably the thiolated biomolecule is a thiolated deoxyribonucleic acid (DNA). Thiolated nucleic acids, including thiolated DNA, are well-known in the art and are described, e.g., in Oh, J W et al., J Am Chem Soc, 2014, 136(40):14052-9 or in Robinson, I et al., Nanoscale, 2010, 2(12):2624-30.

The thiolated biomolecule which is used in step (iv) of the method according to this fifth aspect of the invention is as defined in step (i). Typically, it is preferred that the same thiolated biomolecule (or the same mixture of two or more thiolated biomolecules) is used both in step (i) and in step (iv), so that a self-assembled monolayer of the same thiolated biomolecule is obtained on the complete surface of the metal nanoparticle NP1. However, it is also possible to use different thiolated biomolecules, or different mixtures of two or more thiolated biomolecules, in steps (iii) and (iv).

In a sixth aspect, the invention relates to a functionalized nanoparticle which is obtainable by (or obtained by) the method of the fifth aspect of the invention.

The products provided in accordance with the present invention, including in particular the dimeric nanoparticle assembly, the core-satellite nanoparticle assembly, or the functionalized nanoparticle described herein, can be used for various applications, e.g., for plasmonic applications, including surface plasmon resonance spectroscopy or plasmonic spectroscopy, such as, e.g., surface-enhanced Raman scattering/spectroscopy (SERS) or surface-enhanced fluorescence spectroscopy, and also for optical imaging techniques, photothermal therapy, and as catalysts. The present invention specifically relates to the use of the products provided herein, including the dimeric nanoparticle assembly, the core-satellite nanoparticle assembly, or the functionalized nanoparticle according to the invention, for each one of these applications. Thus, for example, the present invention relates to the use of the dimeric nanoparticle assembly according to the second aspect of the invention, the core-satellite nanoparticle assembly according to the fourth aspect, or the functionalized nanoparticle according to the sixth aspect in plasmonic spectroscopy, particularly in surface-enhanced Raman spectroscopy. Accordingly, the invention relates to the use of the dimeric nanoparticle assembly according to the second aspect, the core-satellite nanoparticle assembly according to the fourth aspect, or the functionalized nanoparticle according to the sixth aspect as a marker in plasmonic spectroscopy, particularly as a SERS marker. The present invention likewise provide a spectroscopic marker, particularly a SERS marker, wherein the spectroscopic marker or the SERS marker comprises (or, preferably, consists of) the dimeric nanoparticle assembly according to the second aspect, or the core-satellite nanoparticle assembly according to the fourth aspect, or the functionalized nanoparticle according to the sixth aspect.

Moreover, the products according to the present invention, including in particular the dimeric nanoparticle assembly, the core-satellite nanoparticle assembly, or the functionalized nanoparticle provided herein, can be used, e.g., in diagnostics, in immunoassays, in flow cytometry, in high-throughput screening, in DNA/RNA assays, in microarrays, in proteomics, for imaging, for labelling and/or detection, for analyses of blood or tissue samples, for biomedical imaging, for immuno-SERS microscopy, for tissue-based cancer diagnosis using antibodies labeled with a nanoparticle assembly or functionalized nanoparticle according to the invention, as a document security marker, etc., including also any of the uses/applications described in U.S. Pat. No. 8,854,617. SERS-active assemblies like dimers or core-satellites according to the present invention, which can produce strong and stable signals, are valuable alternatives for fluorescence techniques that suffer from photo-bleaching and -blinking and find versatile applications.

A corresponding exemplary application is illustrated in FIG. 17 and is further described in the following. Recombinant protein, printed on a test kit made of porous cellulose membrane, is an antigen. When a drop of blood is dropped on the kit, antibody in blood will capture the antigen on the membrane. Here, for example, the antigen is the protein shell of HCV. If a subject (e.g., a human) is infected by HCV, his body produces antibodies. Then, when he drops his blood on the kit having the antigen which is the HCV shell, the antibody in his blood will stay (capturing the antigen) on the kit and other agents in his blood will sink through pore of membrane. Thus, if this antibody can be detected on the kit, it can be concluded that the subject is infected with HCV (or is not infected if the antibody cannot be detected). In accordance with the present invention, the antibody on the kit can be detected by using SERS technique. Thus, a SERS-active platform like dimer or core-satellite that produce strong SERS signal is needed. In FIG. 17, a symmetric core-satellite whose 4-nitrothiophenols (NTPs, the role is “SERS reporter”) are functionalized on satellite surface is used. To interact the SERS-active symmetric core-satellites according to the invention and antibodies on the kit, the SERS-active symmetric core-satellites are covered with Protein A that binds the Fc region of antibody (any type of). Finally, when this core-satellites are dropped on the kit, antibody captures the core-satellite. Due to a large extinction coefficient of noble metal NP, the core-satellite can be quickly recognized, in some cases even with the naked eye (see the dark spot in FIG. 17). However, in the early stage of infection, for example, the dark spot stained by core-satellite may be pale or invisible due to the low concentration of antibodies in a drop of blood. In this case, SERS can be measured on the blue spot instead of the colorimetric detection. Clearly, SERS signal from NTP on the core-satellite will be observable (see FIG. 17B). Furthermore, the extremely low detection limit (e.g., a detectable SERS signal from a single dimer of the present invention) will offer ultra-high sensitivity. The synthetic method for assemblies or functionalizing monomeric particles with bio-materials, proceeded in colloidal system, enables mass production within a short time.

The following definitions apply throughout the present specification, unless specifically indicated otherwise.

The terms “subjecting” (e.g., “subjecting X to Y”) and “contacting” (e.g., “contacting X with Y”) are used herein synonymously with “reacting” (e.g., “reacting X with Y”).

The terms “allowing” (or related forms like “allow”), as e.g. In the expression “allowing the formation of a self-assembled monolayer”, is used herein synonymously with “inducing” (or related forms like “induce”), such as e.g. “inducing the formation of a self-assembled monolayer”.

The term “sonication” refers to the application of sound energy to a sample, typically at a frequency equal to or greater than about 16 kHz (also referred to as “ultrasound”; e.g., from about 16 kHz to about 200 MHz, preferably from about 20 kHz to about 2 MHz, more preferably from about 25 kHz to about 200 kHz, even more preferably from about 30 kHz to about 100 kHz). Thus, if a method step is to be conducted “by using sonication”, the corresponding step shall carried out while applying sound at any of the above-described frequencies (e.g., in an ultrasonic bath).

The term “organic group” refers to a chemical group containing at least one carbon atom. The term “hydrocarbon group” refers to a group consisting of carbon atoms and hydrogen atoms.

The term “alicyclic” is used in connection with cyclic groups and denotes that the corresponding cyclic group is non-aromatic.

The term “hydrocarbyl” refers to a monovalent hydrocarbon group which may be acyclic (i.e., non-cyclic) or cyclic, or it may be composed of both acyclic and cyclic groups/subunits. An acyclic hydrocarbyl or an acyclic subunit in a hydrocarbyl may be linear or branched, and may further be saturated or unsaturated. A cyclic hydrocarbyl or a cyclic subunit in a hydrocarbyl may be saturated, partially unsaturated (i.e., unsaturated but not aromatic) or aromatic. A “C₁₋₁₀ hydrocarbyl” denotes a hydrocarbyl group having 1 to 10 carbon atoms. Exemplary hydrocarbyl groups include, infer alia, alkyl, alkenyl, alkynyl, cycloalkyl, cycloalkenyl, aryl, or a composite group composed of two or more of the aforementioned groups (such as, e.g., alkylcycloalkyl, alkylcycloalkenyl, alkylarylalkenyl, arylalkyl, or alkynylaryl).

As used herein, the term “alkyl” refers to a monovalent saturated acyclic (i.e., non-cyclic) hydrocarbon group which may be linear or branched. Accordingly, an “alky” group does not comprise any carbon-to-carbon double bond or any carbon-to-carbon triple bond. A “C₁₋₅ alkyl” denotes an alkyl group having 1 to 5 carbon atoms. Preferred exemplary alkyl groups are methyl, ethyl, propyl (e.g., n-propyl or isopropyl), or butyl (e.g., n-butyl, isobutyl, sec-butyl, or tert-butyl). Unless defined otherwise, the term “alkyl” preferably refers to C₁₋₅ alkyl, more preferably to methyl or ethyl, and even more preferably to methyl.

As used herein, the term “alkenyl” refers to a monovalent unsaturated acyclic hydrocarbon group which may be linear or branched and comprises one or more (e.g., one or two) carbon-to-carbon double bonds while it does not comprise any carbon-to-carbon triple bond. The term “C₂₋₅ alkenyl” denotes an alkenyl group having 2 to 5 carbon atoms. Preferred exemplary alkenyl groups are ethenyl, propenyl (e.g., prop-1-en-1-yl, prop-1-en-2-yl, or prop-2-en-1-yl), butenyl, butadienyl (e.g., buta-1,3-dien-1-yl or buta-1,3-dien-2-yl), pentenyl, or pentadienyl (e.g., isoprenyl). Unless defined otherwise, the term “alkenyl” preferably refers to C₂ alkenyl.

As used herein, the term “alkynyl” refers to a monovalent unsaturated acyclic hydrocarbon group which may be linear or branched and comprises one or more (e.g., one or two) carbon-to-carbon triple bonds and optionally one or more (e.g., one or two) carbon-to-carbon double bonds. The term “C₂₋₅ alkynyl” denotes an alkynyl group having 2 to 5 carbon atoms. Preferred exemplary alkynyl groups are ethynyl, propynyl (e.g., propargyl), or butynyl. Unless defined otherwise, the term “alkynyl” preferably refers to C₂₋₄ alkynyl.

As used herein, the term “alkylene” refers to an alkanediyl group, i.e. a divalent saturated acyclic hydrocarbon group which may be linear or branched. A “C-alkylene” denotes an alkylene group having 1 to 5 carbon atoms, and the term “C₁₋₅ alkylene” indicates that a covalent bond (corresponding to the option “C₀ akylene”) or a C₁₋₃ alkylene is present. Preferred exemplary alkylene groups are methylene (—CH₂—), ethylene (e.g., —CH₂—CH₂— or —CH(—CH₃)—), propylene (e.g., —CH₂—CH₂—CH₂—, —CH(—CH₂CH₃)—, —CH₂—CH(—CH₃)—, or —CH(—CH₃)—CH₂—), or butylene (e.g., —CH₂—CH₂—CH₂—CH₂—). Unless defined otherwise, the term “alkylene” preferably refers to C₁₋₄ alkylene (including, in particular, linear C₄ alkylene), more preferably to methylene or ethylene, and even more preferably to methylene.

As used herein, the term “carbocyclyl” refers to a hydrocarbon ring group, including monocyclic rings as well as bridged ring, spiro ring and/or fused ring systems (which may be composed, e.g., of two or three rings), wherein said ring group may be saturated, partially unsaturated (i.e., unsaturated but not aromatic) or aromatic. Unless defined otherwise, “carbocyclyl” preferably refers to aryl, cycloalkyl or cycloalkenyl.

As used herein, the term “heterocyclyl” refers to a ring group, including monocyclic rings as well as bridged ring, spiro ring and/or fused ring systems (which may be composed, e.g., of two or three rings), wherein said ring group comprises one or more (such as, e.g., one, two, three, or four) ring heteroatoms independently selected from O, S and N, and the remaining ring atoms are carbon atoms, wherein one or more S ring atoms (if present) and/or one or more N ring atoms (if present) may optionally be oxidized, wherein one or more carbon ring atoms may optionally be oxidized (i.e., to form an oxo group), and further wherein said ring group may be saturated, partially unsaturated (i.e., unsaturated but not aromatic) or aromatic. For example, each heteroatom-containing ring comprised in said ring group may contain one or two O atoms and/or one or two S atoms (which may optionally be oxidized) and/or one, two, three or four N atoms (which may optionally be oxidized), provided that the total number of heteroatoms in the corresponding heteroatom-containing ring is 1 to 4 and that there is at least one carbon ring atom (which may optionally be oxidized) in the corresponding heteroatom-containing ring. Unless defined otherwise, “heterocyclyl” preferably refers to heteroaryl, heterocycloalkyl or heterocycloalkenyl.

As used herein, the term “aryl” refers to an aromatic hydrocarbon ring group, including monocyclic aromatic rings as well as bridged ring and/or fused ring systems containing at least one aromatic ring (e.g., ring systems composed of two or three fused rings, wherein at least one of these fused rings is aromatic; or bridged ring systems composed of two or three rings, wherein at least one of these bridged rings is aromatic). “Aryl” may, e.g., refer to phenyl, naphthyl, dialinyl (i.e., 1,2-dihydronaphthyl), tetralinyl (i.e., 1,2,3,4-tetrahydronaphthyl), indanyl, indenyl (e.g., 1H-indenyl), anthracenyl, phenanthrenyl, 9H-fluorenyl, or azulenyl. Unless defined otherwise, an “aryl” preferably has 6 to 14 ring atoms, more preferably 6 to 10 ring atoms, even more preferably refers to phenyl or naphthyl, and most preferably refers to phenyl.

As used herein, the term “heteroaryl” refers to an aromatic ring group, including monocyclic aromatic rings as well as bridged ring and/or fused ring systems containing at least one aromatic ring (e.g., ring systems composed of two or three fused rings, wherein at least one of these fused rings is aromatic; or bridged ring systems composed of two or three rings, wherein at least one of these bridged rings is aromatic), wherein said aromatic ring group comprises one or more (such as, e.g., one, two, three, or four) ring heteroatoms independently selected from O, S and N, and the remaining ring atoms are carbon atoms, wherein one or more S ring atoms (if present) and/or one or more N ring atoms (If present) may optionally be oxidized, and further wherein one or more carbon ring atoms may optionally be oxidized (i.e., to form an oxo group). For example, each heteroatom-containing ring comprised in said aromatic ring group may contain one or two O atoms and/or one or two S atoms (which may optionally be oxidized) and/or one, two, three or four N atoms (which may optionally be oxidized), provided that the total number of heteroatoms in the corresponding heteroatom-containing ring is 1 to 4 and that there is at least one carbon ring atom (which may optionally be oxidized) in the corresponding heteroatom-containing ring. “Heteroaryl” may, e.g., refer to thienyl (i.e., thiophenyl), benzo[b]thienyl, naphtho[2,3-b]thienyl, thianthrenyl, furyl (i.e., furanyl), benzofuranyl, isobenzofuranyl, chromanyl, chromenyl (e.g., 2H-1-benzopyranyl or 4H-1-benzopyranyl), isochromenyl (e.g., 1H-2-benzopyranyl), chromonyl, xanthenyl, phenoxathinyl, pyrrolyl (e.g., 1H-pyrolyl), imidazolyl, pyrazolyl, pyridyl (i.e., pyridinyl; e.g., 2-pyridyl, 3-pyridyl, or 4-pyridyl), pyrazinyl, pyrimidinyl, pyridazinyl, indolyl (e.g., 3H-indolyl), isoindolyl, indazolyl, indolizinyl, purinyl, quinolyl, isoquinolyl, phthalazinyl, naphthyridinyl, quinoxalinyl, cinnolinyl, pteridinyl, carbazoyl, β-carbolinyl, phenanthridinyl, acridinyl, perimidinyl, phenanthrolinyl (e.g., [1,10]phenanthrolinyl, [1,7]phenanthrofinyl, or [4,7]phenanthrolinyl), phenazinyl, thiazolyl, isothlazolyl, phenothiazinyl, oxazolyl, isoxazolyl, oxadiazolyl (e.g., 1,2,4-oxadiazolyl, 1,2,5-oxadiazolyl (i.e., furazanyl), or 1,3,4-oxadiazolyl), thiadiazolyl (e.g., 1,2,4-thiadiazolyl, 1,2,5-thiadiazolyl, or 1,3,4-thiadiazolyl), phenoxazinyl, pyrazolo[1,5-a]pyrimidinyl (e.g., pyrazolo[1,5-a]pyrimidin-3-yl), 1,2-benzisoxazol-3-yl, benzothiazolyl, benzothiadiazolyl, benzoxazolyl, benzisoxazolyl, benzimidazolyl, benzo[b]thiophenyl (i.e., benzothienyl), triazolyl (e.g., 1H-1,2,3-triazolyl, 2H-1,2,3-triazolyl, 1H-1,2,4-triazolyl, or 4H-1,2,4-triazolyl), benzotriazolyl, 1H-tetrazolyl, 2H-tetrazolyl, triazinyl (e.g., 1,2,3-triazinyl, 1,2,4-triazinyl, or 1,3,5-triazinyl), furo[2,3-c]pyridinyl, dihydro furopyridinyl (e.g., 2,3-dihydrofuro[2,3-c]pyridinyl or 1,3-dihydrofuro[3,4-c]pyridinyl), imidazopyridinyl (e.g., imidazo[1,2-a]pyridinyl or imidazo[3,2-a]pyridinyl), quinazolinyl, thienopyridinyl, tetrahydrothenopyridinyl (e.g., 4,5,6,7-tetrahydrothieno[3,2-c]pyradinyl), dibenzofuranyl, 1,3-benzodioxolyl, benzodioxanyl (e.g., 1,3-benzodioxanyl or 1,4-benzodioxanyl), or coumarinyl. Unless defined otherwise, the term “heteroaryl” preferably refers to a 5 to 14 membered (more preferably 5 to 10 membered) monocyclic ring or fused ring system comprising one or more (e.g., one, two, three or four) ring heteroatoms independently selected from O, S and N, wherein one or more S ring atoms (if present) and/or one or more N ring atoms (if present) are optionally oxidized, and wherein one or more carbon ring atoms are optionally oxidized; even more preferably, a “heteroaryl” refers to a 5 or 6 membered monocyclic ring comprising one or more (e.g., one, two or three) ring heteroatoms independently selected from O, S and N, wherein one or more S ring atoms (if present) and/or one or more N ring atoms (if present) are optionally oxidized, and wherein one or more carbon ring atoms are optionally oxidized. Moreover, unless defined otherwise, particularly preferred examples of a “heteroaryl” include pyridinyl (e.g., 2-pyridyl, 3-pyridyl, or 4-pyridyl), imidazoyl, thiazolyl, 1H-tetrazolyl, 2H-tetrazolyl, thenyl (i.e., thiophenyl), or pyrimidinyl.

As used herein, the term “cycloalkyl” refers to a saturated hydrocarbon ring group, including monocyclic rings as well as bridged ring, spiro ring and/or fused ring systems (which may be composed, e.g., of two or three rings; such as, e.g., a fused ring system composed of two or three fused rings). “Cycloalkyl” may, e.g., refer to cyclopropyl, cyclobutyl, cyclopentyl, cyclohexyl, cycloheptyl, decalinyl (i.e., decahydronaphthyl), or adamantyl. Unless defined otherwise, “cycloalkyl” preferably refers to a C₃₋₁₁ cycloalkyl, and more preferably refers to a C₃₋₇ cycloalkyl. A particularly preferred “cycloalkyl” is a monocyclic saturated hydrocarbon ring having 3 to 7 ring members. Moreover, unless defined otherwise, particularly preferred examples of a“cycloakyl” include cyclohexyl or cyclopropyl, particularly cyclohexyl.

As used herein, the term “heterocycloalkyl” refers to a saturated ring group, including monocyclic rings as well as bridged ring, spiro ring and/or fused ring systems (which may be composed, e.g., of two or three rings; such as, e.g., a fused ring system composed of two or three fused rings), wherein said ring group contains one or more (such as, e.g., one, two, three, or four) ring heteroatoms independently selected from O, S and N, and the remaining ring atoms are carbon atoms, wherein one or more S ring atoms (if present) and/or one or more N ring atoms (if present) may optionally be oxidized, and further wherein one or more carbon ring atoms may optionally be oxidized (i.e., to form an oxo group). For example, each heteroatom-containing ring comprised in said saturated ring group may contain one or two O atoms and/or one or two S atoms (which may optionally be oxidized) and/or one, two, three or four N atoms (which may optionally be oxidized), provided that the total number of heteroatoms in the corresponding heteroatom-containing ring is 1 to 4 and that there is at least one carbon ring atom (which may optionally be oxidized) in the corresponding heteroatom-containing ring. “Heterocycloalkyl” may, e.g., refer to aziridinyl, azetidinyl, pyrrolidinyl, imidazolidinyl, pyrazolidinyl, piperidinyl, piperazinyl, azepanyl, diazepanyl (e.g., 1,4-diazepanyl), oxazolidinyl, isoxazolidinyl, thiazolidinyl, isothiazolidinyl, morpholinyl (e.g., morpholin-4-yl), thiomorpholinyl (e.g., thiomorpholin-4-yl), oxazepanyl, oxiranyl, oxetanyl, tetrahydrofuranyl, 1,3-dioxolanyl, tetrahydropyranyl, 1,4-dioxanyl, oxepanyl, thiiranyl, thietanyl, tetrahydrothiophenyl (i.e., thiolanyl), 1,3-dithiolanyl, thianyl, thiepanyl, decahydroquinolinyl, decahydroisoquinolinyl, or 2-oxa-5-aza-bicyclo[2.2.1]hept-5-yl. Unless defined otherwise, “heterocycloalky” preferably refers to a 3 to 11 membered saturated ring group, which is a monocyclic ring or a fused ring system (e.g., a fused ring system composed of two fused rings), wherein said ring group contains one or more (e.g., one, two, three, or four) ring heteroatoms independently selected from O, S and N, wherein one or more S ring atoms (if present) and/or one or more N ring atoms (if present) are optionally oxidized, and wherein one or more carbon ring atoms are optionally oxidized; more preferably, “heterocycloalkyl” refers to a 5 to 7 membered saturated monocyclic ring group containing one or more (e.g., one, two, or three) ring heteroatoms independently selected from O, S and N, wherein one or more S ring atoms (if present) and/or one or more N ring atoms (if present) are optionally oxidized, and wherein one or more carbon ring atoms are optionally oxidized. Moreover, unless defined otherwise, particularly preferred examples of a “heterocycloalkyl” include tetrahydropyranyl, piperidinyl, piperazinyl, morpholinyl, pyrrolidinyl, or tetrahydrofuranyl.

As used herein, the term “cycloakenyl” refers to an unsaturated alicyclic (non-aromatic) hydrocarbon ring group, including monocyclic rings as well as bridged ring, spiro ring and/or fused ring systems (which may be composed, e.g., of two or three rings; such as, e.g., a fused ring system composed of two or three fused rings), wherein said hydrocarbon ring group comprises one or more (e.g., one or two) carbon-to-carbon double bonds and does not comprise any carbon-to-carbon triple bond. “Cycloalkenyl” may, e.g., refer to cyclopropenyl, cyclobutenyl, cyclopentenyl, cyclohexenyl, cyclohexadienyl, cycloheptenyl, or cycloheptadienyl. Unless defined otherwise, “cycloalkenyl” preferably refers to a C₃₋₁₁ cycloalkenyl, and more preferably refers to a C₃₋₇ cycloalkenyl. A particularly preferred “cycloalkenyl” is a monocyclic unsaturated alicyclic hydrocarbon ring having 3 to 7 ring members and containing one or more (e.g., one or two; preferably one) carbon-to-carbon double bonds.

As used herein, the term “heterocycloalkenyl” refers to an unsaturated alicyclic (non-aromatic) ring group, including monocyclic rings as well as bridged ring, spiro ring and/or fused ring systems (which may be composed, e.g., of two or three rings; such as, e.g., a fused ring system composed of two or three fused rings), wherein said ring group contains one or more (such as, e.g., one, two, three, or four) ring heteroatoms independently selected from O, S and N, and the remaining ring atoms are carbon atoms, wherein one or more S ring atoms (if present) and/or one or more N ring atoms (if present) may optionally be oxidized, wherein one or more carbon ring atoms may optionally be oxidized (i.e., to form an oxo group), and further wherein said ring group comprises at least one double bond between adjacent ring atoms and does not comprise any triple bond between adjacent ring atoms. For example, each heteroatom-containing ring comprised in said unsaturated alicyclic ring group may contain one or two O atoms and/or one or two S atoms (which may optionally be oxidized) and/or one, two, three or four N atoms (which may optionally be oxidized), provided that the total number of heteroatoms in the corresponding heteroatom-containing ring is 1 to 4 and that there is at least one carbon ring atom (which may optionally be oxidized) in the corresponding heteroatom-containing ring. “Heterocycloalkenyl” may, e.g., refer to imidazolinyl (e.g., 2-Imidazolinyl (i.e., 4,5-dihydro-1H-imidazolyl), 3-imidazolinyl, or 4-imidazolinyl), tetrahydropyridinyl (e.g., 1,2,3,6-tetrahydropyridinyl), dihydropyridinyl (e.g., 1,2-dihydropyridinyl or 2,3-dihydropyridinyl), pyranyl (e.g., 2H-pyranyl or 4H-pyranyl), thiopyranyl (e.g., 2H-thiopyranyl or 4H-thiopyranyl), dihydropyranyl, dihydrofuranyl, dihydropyrazolyl, dihydropyrazinyl, dihydroisoindolyl, octahydroquinolinyl (e.g., 1,2,3,4,4a,5,6,7-octahydroquinolinyl), or octahydroisoquinolinyl (e.g., 1,2,3,4,5,6,7,8-octahydroisoquinolinyl). Unless defined otherwise, “heterocycloalkenyl” preferably refers to a 3 to 11 membered unsaturated alicyclic ring group, which is a monocyclic ring or a fused ring system (e.g., a fused ring system composed of two fused rings), wherein said ring group contains one or more (e.g., one, two, three, or four) ring heteroatoms independently selected from O, S and N, wherein one or more S ring atoms (if present) and/or one or more N ring atoms (if present) are optionally oxidized, wherein one or more carbon ring atoms are optionally oxidized, and wherein said ring group comprises at least one double bond between adjacent ring atoms and does not comprise any triple bond between adjacent ring atoms; more preferably, “heterocycloalkenyl” refers to a 5 to 7 membered monocyclic unsaturated non-aromatic ring group containing one or more (e.g., one, two, or three) ring heteroatoms independently selected from O, S and N, wherein one or more S ring atoms (if present) and/or one or more N ring atoms (if present) are optionally oxidized, wherein one or more carbon ring atoms are optionally oxidized, and wherein said ring group comprises at least one double bond between adjacent ring atoms and does not comprise any triple bond between adjacent ring atoms.

As used herein, the term “halogen” refers to fluoro (—F), chloro (—C), bromo (—Br), or iodo (—I).

As used herein, the term “haloalkyl” refers to an alkyl group substituted with one or more (preferably 1 to 6, more preferably 1 to 3) halogen atoms which are selected independently from fluoro, chloro, bromo and iodo, and are preferably all fluoro atoms. It will be understood that the maximum number of halogen atoms is limited by the number of available attachment sites and, thus, depends on the number of carbon atoms comprised in the alkyl moiety of the haloalkyl group. “Haloalkyl” may, e.g., refer to —CF₃, —CHF₂, —CH₂F, —CF₂—CH₃, —CH₂—CF₃, —CH₂—CHF₂, —CH₂—CF₂—CH₃, —CH₂—CF₂—CF₃, or —CH(CF)₂. A particularly preferred “haloalkyl” group is —CF₃.

As used herein, the terms “optional”, “optionally” and “may” denote that the indicated feature may be present but can also be absent. Whenever the term “optional”, “optionally” or “may” is used, the present invention specifically relates to both possibilities, i.e., that the corresponding feature is present or, alternatively, that the corresponding feature is absent. For example, the expression “X is optionally substituted with Y” (or “X may be substituted with Y”) means that X is either substituted with Y or is unsubstituted. Likewise, if a component of a composition is indicated to be “optional”, the invention specifically relates to both possibilities, i.e., that the corresponding component is present (contained in the composition) or that the corresponding component is absent from the composition.

Various groups are referred to as being “optionally substituted” in this specification. Generally, these groups may carry one or more substituents, such as, e.g., one, two, three or four substituents. It will be understood that the maximum number of substituents is limited by the number of attachment sites available on the substituted moiety. Unless defined otherwise, the “optionally substituted” groups referred to in this specification carry preferably not more than two substituents and may, in particular, carry only one substituent. Moreover, unless defined otherwise, it is preferred that the optional substituents are absent, i.e. that the corresponding groups are unsubstituted.

A skilled person will appreciate that the substituent groups comprised in the compounds described herein may be attached to the remainder of the respective compound via a number of different positions of the corresponding specific substituent group. Unless defined otherwise, the preferred attachment positions for the various specific substituent groups are as illustrated in the examples.

The term “nucleic acid” is well known in the art and refers, in particular, to all forms of naturally occurring or recombinantly generated types of nucleic acids and/or nucleotide sequences as well as to chemically synthesized nucleic acids/nucleotide sequences. This term also encompasses nucleic acid analogs and nucleic acid derivatives such as locked DNA, PNA, oligonucleotide thiophosphates and substituted ribo-oligonucleotides. Furthermore, the term “nucleic acid” also refers to any molecule that comprises nucleotides or nucleotide analogs. Preferably, the term “nucleic acid” refers to deoxyribonucleic acid (DNA) and/or ribonucleic acid (RNA). DNA and RNA may optionally comprise unnatural nucleotides and may be single or double stranded.

As used herein, unless explicitly indicated otherwise or contradicted by context, the terms “a”, “an” and “the” are used interchangeably with “one or more” and “at least one”. Thus, for example, a composition comprising “a” compound of formula (I) can be interpreted as referring to a composition comprising “one or more” compounds of formula (I).

As used herein, the term “about” preferably refers to ±10% of the indicated numerical value, more preferably to ±5% of the indicated numerical value, and in particular to the exact numerical value indicated. If the term “about” is used in connection with the endpoints of a range, it preferably refers to the range from the lower endpoint −10% of its indicated numerical value to the upper endpoint +10% of its indicated numerical value, more preferably to the range from of the lower endpoint −5% to the upper endpoint +5%, and even more preferably to the range defined by the exact numerical values of the lower endpoint and the upper endpoint. If the term “about” is used in connection with the endpoint of an open-ended range, it preferably refers to the corresponding range starting from the lower endpoint −10% or from the upper endpoint +10%, more preferably to the range starting from the lower endpoint −5% or from the upper endpoint +5%, and even more preferably to the open-ended range defined by the exact numerical value of the corresponding endpoint. If the term “about” is used in connection with a parameter that is quantified in integers, such as the number of nucleotides in a given nucleic acid, the numbers corresponding to ±10% or ±5% of the indicated numerical value are to be rounded to the nearest integer (using the tie-breaking rule “round half up”).

As used herein, the term “comprising” (or “comprise”, “comprises”, “contain”, “contains”, or “containing”), unless explicitly indicated otherwise or contradicted by context, has the meaning of “containing, inter alia”, i.e., “containing, among further optional elements, . . . ”. In addition thereto, this term also includes the narrower meanings of “consisting essentially of” and “consisting of”. For example, the term “A comprising B and C” has the meaning of “A containing, inter alia, B and C”, wherein A may contain further optional elements (e.g., “A containing B, C and D” would also be encompassed), but this term also includes the meaning of “A consisting essentially of B and C” and the meaning of “A consisting of B and C” (i.e., no other components than B and C are comprised in A).

Unless specifically indicated otherwise, all properties and parameters referred to herein (including, e.g., any amounts/concentrations indicated in “mg/ml”, in “% (w/v)” (i.e., mg/100 μl), in “% (v/v)”, or in vol-% (i.e., % (v/v)), as well as any pH values) are preferably to be determined at standard ambient temperature and pressure conditions, particularly at a temperature of 25° C. (298.15 K) and at an absolute pressure of 101.325 kPa (1 atm).

The different method steps of the methods described/provided herein can, in general, be carried out in any suitable order, unless indicated otherwise or contradicted by context, and are preferably carried out in the specific order in which they are indicated.

It is to be understood that the present invention specifically relates to each and every combination of features and embodiments described herein, including any combination of general and/or preferred features/embodiments. In particular, the invention specifically relates to each combination of meanings (including general and/or preferred meanings) for the various groups and variables comprised in formula (I).

In this specification, a number of documents including patent applications, scientific literature and manufacturers' manuals are cited. The disclosure of these documents, while not considered relevant for the patentability of this invention, is herewith incorporated by reference in its entirety. More specifically, all referenced documents are incorporated by reference to the same extent as if each individual document was specifically and individually indicated to be Incorporated by reference.

The reference in this specification to any prior publication (or information derived therefrom) is not and should not be taken as an acknowledgment or admission or any form of suggestion that the corresponding prior publication (or the information derived therefrom) forms part of the common general knowledge in the technical field to which the present specification relates.

The invention is also described by the following illustrative figures. The appended figures show:

FIG. 1: (A) Scanning and (B) transmission electron microscopy image of quasi-ideal dimers prepared by the substrate-based sequential dimer assembly method (see Example 1).

FIG. 2: (A) Unpolarized single-particle scattering spectra from randomly selected quasi-ideal dimers linked by C8. Vertical dotted lines are guides to the eye. (B) Simulated scattering spectra of a modeled dimer. Bottom and upper gray fines correspond to a transverse and longitudinal excitation, respectively. Distinct band positions are indicated. The black line that is the sum of two gray lines corresponds to an unpolarized excitation. See Example 1.

FIG. 3: (A) Scattering spectra from a selected quasi-Ideal dimer as a function of the polarizer angle and (B) a polar plot of scattering intensities at 734 nm (see Example 1).

FIG. 4: (A) Experimental and (B) simulated extinction spectra of dimers having different gap distances (see Example 1).

FIG. 5: (A) A TEM image and (B) size distribution of AuNSs prepared by etching. (C) Dark-field scattering spectra from isolated AuNSs. The dotted line is drawn vertically for guiding the eye. See Example 1.

FIG. 6: Substrate-based sequential dimer assembly method (see Example 1).

FIG. 7: (A) A representative SEM image of assemblies formed on a glass substrate and (B) histogram showing the proportions of assembly types, measured from SEM images obtained in six independent experiments (see Example 1).

FIG. 8: (A) A representative SEM image and (B) histogram of gold nanocube dimers 35 prepared in accordance with the invention.

FIG. 9: Scheme illustrating the preparation of ideal dimeric nanoparticle assemblies according to the present invention.

FIG. 10: UV/vis extinction spectra from core-satellites. Satellite sizes are varied to 17, 24, and 30 nm in diameter (see Example 2).

FIG. 11: TEM images of core-satellites corresponding to FIG. 10. (A) and (B) 17 nm satellites, (C) and (D) 24 nm satellites, and (E) and (F) 30 nm satellites.

FIG. 12: SEM image of asymmetric AuNS core-AuNP satellites (see Example 1).

FIG. 13: SEM image of asymmetric AuNC core-AuNP satellites (see Example 1).

FIG. 14: Results of stability tests (see Example 2).

FIG. 15: (A) SERS spectra from a selected quasi-Ideal dimer as a function of the polarizer angle and (B) a polar plot of SERS intensities at 1175 cm⁻¹.

FIG. 16: General scheme showing (1) the efficient removal of a detergent bilayer (for illustration, a CTA⁺ bilayer is shown) from the surface of metal nanoparticles fixed on substrate or dispersed in solvent, (2) the further molecular functionalization, and (3) assembly.

FIG. 17: The antibody detection result performed on flow assay test kit comprised of porous cellulose membrane where recombinant proteins are printed. (A) Photograph. A dark spot (which is originally blue colored) is observed on the test group. (B) SERS spectrum measured from the blue spot on the test group. (C) Schematic figure corresponding to the blue spot on the test group.

FIG. 18: CCD camera images (at 10 ms exposure) of (A) and (C) monomeric and (B) and (D) dimeric AuNSs. These images are taken on a home-built modified nanoparticle tracking setup that allows (A) and (B) Rayleigh and (C) and (D) Raman channel in parallel. For better showing, white and black circles are added in the case of AuNS dimer. See Example 3.

FIG. 19: Photograph of DNA-functionalized AuNS run on agarose gel. The position of AuNS on the gel is indicated by gray bands (which were originally red colored). White curved lines remark the gray bands. A schematic representation of the corresponding DNA-functionized AuNS is shown above the photograph of the gel. See Example 4.

FIG. 20: UV-vis extinction spectra of symmetric core-satellite assemblies prepared by seven Independent experiments (see Example 5).

FIG. 21: Normalized UV-vis extinction spectra (top) and SERS spectra (bottom) of core-satellite assemblies having NTP molecules either on the satellite or in the gap (see Example 6).

FIG. 22: SERS spectra of core-satellite assemblies having different Raman-active molecules in the gap. The following Raman-active molecules are used: 4-nitrothiophenol (NTP), 7-mercapto-4-methylcoumarin (MMC), thio-2-naphthol (TN), 2,3,5,6-tetrafluoro-4-mercaptobenzoic acid (TFMBA), mercapto-4-methyl-5-thioacetic acid (MMTA), 2-bromo-4-mercaptobenzoic acid (BMBA), ethyl(2E,4E,6E,8E,10E,12E,14E)-15-(4-(tert-butylthio)phenyl)pentadeca-2,4,6,8,10,12,14-heptanoate (Polyene 7DB), and ethyl(2E,4E)-5-(4-(tert-butylthio)phenyl)penta-2,4-dienoate (Polyene 2DB). See Example 6.

FIG. 23: Normalized UV-vis extinction spectra and SEM images of quasi-ideal core-satellite assemblies whose satellites are functionalized with either MUA or MPA (see Example 7).

The invention will now be described by reference to the following examples which are merely illustrative and are not to be construed as a limitation of the scope of the present invention.

### EXAMPLES

### Example 1: Ideal Dimers of Gold Nanospheres Linked by a C6, C8 or C10 SAM or a Raman-Active Polyene Dithiol, Ideal Dimers of Gold Nanocubes, and Asymmetric Core-Satellite Assemblies

A pair of two spherical nanoparticles (NPs), a dimer, has been a valuable model to study surface plasmon (SP) coupling due to its structural simplicity like a diatomic molecule (Sheikholeslami, S et al., Nano Lett 2010, 10, 2655-2660). According to the plasmon hybridization model analogous to molecular orbital theory, a symmetric dimer allows just one bright mode when the linearly polarized light is applied to the dimer axis parallel or perpendicular (Nordlander, P et al., Nano Let 2004, 4, 899-903). It implies that the use of dimers greatly reduces the complexity and difficulty in the result interpretation. This has 35 encouraged both theorists and experimentalists to prefer dimers. However, the intrinsic structural non-ideality of experimental dimers constructed by irregular gap distances and polyhedral NPs has disrupted the accurate comparison between theoretical and experimental results (Popp, P S at al., Small 2016, 12, 1667-1675). For this reason, researchers have made their efforts to enhance the ideality of experimental dimers by discarding the variations either in the building block or the gap distance (Tian, X et al., J. Phys. Chem. C 2014, 118, 13801-13808; Cha, H et al., ACS Nano 2014, 8, 8554-8563; Craci, C et al., Science 2012, 337, 1072-1074). However, in spite of the reduced non-ideality, such partially idealized dimers are not appropriate for precision plasmonics owing to inevitably quite broad spectral deviations at the single-NP level. In particular, various gap morphologies that are unavoidably created in dimers composed of polyhedral NPs produce disparate SP coupling energies largely deviated from the simulation results, albeit with similar gap distances (Popp, P S at al., Small 2016, 12, 1667-1675).

The present invention provides a novel assembly method producing highly desired ideal dimers in nearly 87% yield. Since the reduction-based bottom-up methods offer faceted NPs with relatively large size distributions, the inventors prepared isotropic monodisperse gold nanospheres (AuNSs) by means of the chemical etching method to get ready for ideal dimer assembly (see materials and methods further below). Etchants preferentially remove the atoms at the vertices and edges of anisotropic NPs due to high surface energy there (Rodriguez-Fernández, J et al. J. Phys. Chem. B 2005, 109, 14257-14261; Lee, Y-J et al., ACS Nano 2013, 7, 11064-11070; Ruan, Q et al., Adv. Opt. Mater. 2014, 2, 65-73). As a result, the etched NPs get a smooth surface and high sphericity. The prepared AuNSs (50±2.5 nm) display the extremely uniform single-particle dark-field (DF) scattering spectra (see FIG. 5). This spectral homogeneity is accomplished by not only the narrow size distribution but also the isotropy. Next, the inventors conducted the sequential dimer assembly process on a glass substrate to avoid the aggregation during introducing a well-ordered alkanedithiol self-assembled monolayer (SAM) in the gap (see materials and methods further below). In the protocol provided herein, the electrostatic interaction of a pure glass substrate and positively charged AuNSs capped by cetyltrimethylammonium bromide (CTAB) bilayer is regulated by the use of water and acetonitrile (Ruan, Q at al., Adv. Opt Mater. 2014, 2, 65-73). Solvent kinds determine the dissociation of the silanol groups (Si—OH to Si—O—) on the glass substrate (Behrens, S H et al., J. Chem. Phys. 2001, 115, 6716-6721). Ultimately, the negative surface charge density of glass substrate is controllable as a function of solvent kinds. Perfectly removing the CTAB bilayer on NPs has been a chalenging task but is crucial to form a SAM in the gap. The concept of critical micelle concentration (CMC) leads the inventors to use organic solvent. In principle, organic solvents such as ethanol and acetonitrile raise the CMC of CTAB, thus the destabilized CTAB bilayer can easily be substituted by thiolated molecules (indrasekara, ASDS t al., Part. Part Syst. Charact. 2014, 31, 819-838). However, since the destabilized CTAB bilayer that is still quite robust results in the incomplete substitution, the formation of dimer does not occur. Sonication during the incubation is a known method for efficient CTAB replacement (Tebbe, M et al., ACS Appl. Mater. Interfaces 2015, 7, 5984-5991). Unfortunately, this sonication method that induces a detachment of AuNSs from substrate is not applicable in the present assembly method. Instead, the inventors additionally added NaBr that unsettles a gathering of CTAB molecules (Hayes, P L et al., J. Phys. Chem. B 2010, 114, 4495-4502). Thus, the mixture of NaBr, thiolated molecules, and an organic solvent synergistically degrades the CTAB bilayer. This key strategy in the protocol according to the invention is demonstrated by dimers linked with hexanedithiol (C6), octanedithiol (C8), and decanedithiol (C10) SAM, producing different SP coupling energies (see FIG. 4). Furthermore, the present assembly method is expandable to CTAB-capped anisotropic NPs like gold nanocubes (see materials and methods further below, and FIG. 8). Gold nanocube dimer is really intriguing because of the large gap area that has been rarely explored (Esteban, R et al., ACS Photonics 2015, 2, 295-305).

As also shown in FIG. 1, the dimers linked by C8 SAM were prepared in high yield and unprecedented ideality. Here the isotropy of monodisperse AuNSs permits the homogeneous gap morphology. In addition, the quasi-crystallinity of the well-ordered molecular SAM enables the gap distance to be regular and constant (Ciracl, C et al., Science 2012, 337, 1072-1074; Love, J C at al., Chem. Rev. 2005, 105, 1103-1169). This structural uniformity of the quasi-ideal dimers according to the invention is reflected in extremely similar spectral shapes of single-particle DF scattering spectra collected under unpolarized light (see FIG. 2A) (Lee, Y-J et al., ACS Nano 2013, 7, 11064-11070). The achieved spectral and structural precisions do not require further correlation analysis of optical and structural properties, which is essential for non-ideal dimers (Popp, P S et al., Small 2016, 12, 1687-1675; Marhaba, S et al., J. Phys. Chem. C 2009, 113, 4349-4356; Yang, L et al., ACS Nano 2016, 10, 1580-1588). Thus, polarization-dependent DF scattering spectroscopy is sufficient to confirm the existence and orientation of dimers (see FIG. 3).

The scattering spectrum simulated with finite-difference time-domain (FDTD) method well reproduces the unpolarized DF scattering spectrum of the quasi-Ideal dimer (see FIG. 2). Since the FDTD simulation allows only one polarization angle of the plane wave source, it is represented by the sum of two scattering spectra simulated through applying the polarized plane wave perpendicular (E_{⊥}) or parallel (E_{∥}) with respect to the axis of modeled dimer. The bands are assigned to longitudinal bonding octupole-octupole (LOP), quadrupole-quadrupole (LOP), dipole-dipole plasmon (LDP), and transverse antibonding dipole-dipole plasmon (TDP) coupling modes, marked as square, triangle, circle, and empty circle, respectively (Zhang, P et al., Phy. Rev. B: Condens. Mater Mater. Phys. 2014, 90, 161407(R); Lassiter, J B et al., Nano Lett. 2008, 8, 1212-1218; Lermé, J, J. Phys. Chem. C 2014, 118, 28118-28133). Although the used NP size is 50 nm that does not show higher surface plasmon resonance modes, the dimer shows higher-order coupling modes (LOP and LQP) in both experiment and simulation. This is because the dipolar oscillator strength contributes to the quadrupolar and octupolar modes (Lermé, J, J. Phys. Chem. C 2014, 118, 28118-28133). The inventors found that the DF scattering intensity at shorter wavelength is contributed by the overlap of TDP and higher-order coupling modes (see FIG. 2B) and their contributions are distinguishable in polarization-resolved DF scattering spectra (see FIG. 3) (Lassiter, J B et al., Nano Let. 2008, 8, 1212-1218). It is known that the electron beam induces a damage of the molecular SAM in the gap during the electron microscopy and it results in the spectral changes although the structural deformation of dimer is evaded under the lowered acceleration voltage condition (Wustholz, K L et al., J. Am. Chem. Soc. 2010, 132, 10903-10910; Henry, A-I et al., J. Phys. Chem. C 2011, 115, 9291-9305; Benz, F et al., J. Phys. Chem. Lett. 2016, 7, 2264-2269). Hence, in simulation, the previously reported value is taken to define the gap distance (Yoon, J H et al., ACS Nano 2012, 6, 7199-7208). The reliability of taken gap distance values is confirmed with the well matched simulated and experimental extinction spectra of quasi-Ideal dimers linked by C6, C8, and C10 SAM (see FIG. 4).

Materials and Methods

1. Synthesis and Characterization of Quasi-Ideal Monomers and Dimers

Materials: Gold(III) chloride trihydrate (HAuCl₄.3H₂O, ≥99.9%, Aldrich), cetyltrimethylammonium bromide (CTAB, ≥96%, Fluka), cetyltrimethylammonium chloride (CTAC, >95.0%, TCI), sodium borohydride (NaBH₄, 96%, Aldrich), ascorbic acid (AA, 99.0%, AppliChem), 1,6-hexanedithiol (C6, 96%, Aldrich), 1,8-octanedithiol (C8, ≥97.0%, TCI), 1,10-decanedithiol (C10, >98.0%, TCI), (11-mercaptoundecyl)-N,N,N-trimethylammonium bromide (MUTAB, ≥90.0%, Aldrich), sodium bromide (NaBr, ≥99.5%, Aldrich), ethanol (EtOH, HPLC grade, Fisher Scientific), acetonitrile (MeCN, HPLC grade, Fisher Scientific), and RBS detergent solution (35 concentrate, Aldrich). All chemicals were used as received. Deionized water (resistivity of 18 MΩ·cm) was prepared using a Millipore Milli-Q system.

Gold nanosphere preparation: In order to achieve a high sphericity and size monodispersity, homogeneous anisotropic gold nanocubes synthesized by seeded growth method were treated by etching (see FIG. 5). The etching process is described in more detail in the section AuNS preparation further below.

In principle, any type of anisotropic NPs (including rod-shaped NPs which are extremely anisotropic) can be used to prepare AuNSs. For example, AuNSs can be prepared by etching polyhedral AuNPs, as described in more detail further below.

First, polyhedral AuNPs were prepared by seeded growth method. Then, prepared polyhedral AuNPs were etched to produce quasi-Ideal spherical AuNPs (AuNSs; gold nanospheres). See also the corresponding protocol described in Ruan, Q et al., Adv. Opt. Mater. 2014, 2, 65-73, which was modified for the present experiment.

Seeds

HAuCl₄ solution (10 mM, 0.25 mL) is first mixed with a CTAB solution (100 mM, 9.75 mL), followed by the rapid injection of a freshly-prepared (ice-cold) NaBH₄ solution (10 mM, 0.60 mL). The resultant was stirred for 1 min and was left undisturbed for 3 h at 30° C. These initial seeds are size-polydisperse, 3.5-7.0 nm in diameter (Dovgolevsky, E et al., Small 2008, 4, 2059-2066; Langille, M R et al., J. Am. Chem. Soc. 2012, 134, 14542-14554).

Polyhedral AuNPs

0.06 mL of the prepared seed solution was injected into a growth solution made of CTAB (100 mM, 4.88 mL), water (95 mL), HAuCl₄ (10 mM, 2 mL), and ascorbic acid (100 mM, 7.5 mL). The mixture was allowed to stir gently and then kept undisturbed for 3 h at 30° C. The grown AuNPs (ca. 30 nm) were concentrated by centrifugation (11000 rpm, 30° C., 40 min) and redispersed in 25 mL of water for second growth process. 9 mL of the grown AuNP solution were added into a CTAC solution (25 mM, 180 mL). After the sequential addition of ascorbic acid (100 mM, 4.5 mL) and HAuCl₄ (10 mM, 9.0 mL), the mixture solution was kept undisturbed for 3 h at 30° C. The obtained further grown polyhedral AuNPs are centrifuged (3000 rpm, 60 min) and redispersed in a CTAB solution (20 mM, 30 mL).

AuNSs

30 mL of the prepared solution of polyhedral AuNPs (ca. 53 nm) were diluted in a CTAB solution (20 mM, 300 mL). Then, a HAuCl₄ solution (10 mM, 1.080 mL) was added under mild stirring at 45° C. After 2 h, the resulted AuNSs (50.0±2.5 nm) were washed by centrifugation twice. In the first round centrifugation (3000 rpm, 45 min), the supernatant was removed and the precipitate (ca. 300 μL) was redispersed in 1.5 mL of water. In the second round centrifugation (2500 rpm, 35 min), the precipitate (ca. 80 μL) was redispersed in 1.6 mL of water.

AuNSs are thus capped by CTAB bilayer (Gómez-Graña, S et al., Langmuir 2012, 28, 1453-1459). The expected AuNS concentration is 11.6 nM. The expected total CTAB concentration (CTAB on AuNSs+free CTAB in solution) in the AuNS solution is 144 μM. When this AuNS solution is diluted to get 5 μM AuNS, the total CTAB concentration will be 62 nM. At this extremely low CTAB concentration, the CTAB bilayer on AuNS degrades and AuNSs immediately aggregate. Hence, to keep the CTAB bilayer on AuNS, the total CTAB concentration in the AuNS solution must be above a certain value. However, too much free CTAB molecules which adsorb on negatively charged substrate fully occupy the glass surface, so that CTAB-capped AuNSs cannot adsorb on the glass surface. Consequently, too low or too high CTAB concentration decreases the efficiency of the 1^{st} NP adsorption on glass (Guo, L et al., Biosens. Bioelectron. 2011, 26, 2246-2251). This concentration range was found to be 1.5-10 μM for AuNS attachment on glass.

Second, AuNCs were prepared by the anisotropic growth of seeds. The protocol described in Dovgolevsky, E et al., Small 2008, 4, 2059-2066 was modified.

Seeds

A HAuCl₄ solution (10 mM, 25 μL) was first mixed with a CTAB solution (100 mM, 750 μL), followed by the rapid injection of a freshly-prepared (Ice-cold) NaBH₄ solution (10 mM, 60 μL). The resultant was stirred for 1 min and was left undisturbed for 1 h at 30° C. Seeds here are basically same with the seeds above (except the concentration and the scale). Aging time does not affect the seed properties.

AuNCs

CTAB (100 mM, 25.6 mL), HAuCl₄ solution (10 mM, 3.2 mL), and ascorbic acid (100 mM, 15.2 mL) were successively added in 128 mL of water to prepare a growth solution. Next, 80 μL of the 10-times diluted seed solution was added in the prepared growth solution under gentie shaking and then the mixture was kept undisturbed at 30° C. After 4 h, the seeded growth process was terminated and the grown AuNCs (51.2±7.3 nm) were washed by centrifugation twice. In the first round centrifugation (4000 rpm, 40 min), the supernatant was removed and the precipitate (ca. 800 μL) was redispersed in 6.4 mL of water. In the second round centrifugation (3000 rpm, 20 min), the precipitate (ca. 120 μL) was redispersed in 3 mL of water. AuNCs are thus capped by CTAB bilayer. The expected AuNC concentration is 586 pM. The expected total CTAB concentration in the AuNC solution is 66 μM.

The centration of prepared AuNSs was calculated with the reported relation of the particle size and the extinction coefficient.

Dimer assembly: For the interaction of a glass slide and AuNSs, a glass slide (25 mm×12 mm) that is cleaned with a hot RBS solution (15%, 90° C.) was immersed in a CTAB solution (5 μM, 5 mL) containing AuNSs (5 μM) for 17 h. Afterward, sequential dimer assembly was performed step by step at 30° C.

The substrate-based sequential dimer assembly method is illustrated in FIG. 6 and comprises the following steps:
Step 1: The glass slide where AuNSs are adsorbed on was washed with water and EtOH and then it was soaked into an alkanedithiol ethanolic solution (1 mM, 5 mL) mixed with NaBr (1 mM) for 1 h. For the polyene dithiol (HS—C₆H₄—CH═CH—CH═CH—C₆H₄—SH, newly synthesized via a Wittig reaction between an aldehyde and triphenylphosphine; both thiol termini may need to be protected by sterically demanding tBu groups), the same concentration (1 mM), but a 1:1 mixture of dichloromethane (DCM) and methanol was used as a solvent.
Step 2: The residual alkanedithiol and NaBr were rinsed away using EtOH and then the glass slide was dipped into 5 mL of MeCN containing AuNSs (20 μM) and NaBr (200 μM) and for 5 h. Here, the added AuNSs do not interact with the exposed surface of the glass slide.
Step 3: The residual NaBr and unbound AuNSs onto the pre-resident AuNSs were removed away using EtOH. Next, the washed glass was immersed in a MUTAB (1 mM) and NaBr (1 mM) ethanolic mixture (5 mL).
Step 4: After 1 h, residual MUTAB and NaBr were washed with EtOH. Then, the glass slide was transferred into a MUTAB ethanolic solution (10 μM, 5 mL). Finally, dimers were detached away from the glass slide by sonication (for 30 s).

The inventors did the stability test of the solution of the 2^{nd} AuNS (20 μM in MeCN) with respect to the NaBr concentration (0-1000 μM). They observed that the too much NaBr concentration induces sticking of AuNS onto the container's wall and too less NaBr concentration induces a fast aggregation. AuNSs are stable in the range between 100 and 500 μM. Next, they tested the dimer yield using the 2^{nd} AuNS solution whose the NaBr concentration is 100, 500, and 1000 μM. As can be seen from the results shown in FIG. 14, extinction decrease at 682 nm but increase at 741 nm are observed in UV-vis spectra. And the increasing higher order structure (dominantly trimer) formation is seen in SEM images. With this correlative tendency, the inventors found an optimal NaBr concentration range (100-300 NM) for high-yield dimer formation.

Gold nanocube dimers preparation: Gold nanocube dimers (AuNC dimers) were prepared as described above for the gold nanosphere dimers (AuNS dimers), and as illustrated in FIG. 6, except that the conditions in the first NP attachment and “step 2” were slightly different. These differences between the preparation of AuNC dimers and AuNS dimers are further described in the following: In the first AuNC attachment step (before “step 1”), a CTAB solution (5 μM, 5 ml) containing AuNCs (2.5 pM) is used for 17 h. In “step 2”, 5 mL of EtOH containing AuNCs (5.0 pM) and NaBr (55 pM) is used for 12 h.

Asymmetric Sphere Core-Sphere Satellites and Asymmetric Cube Core-Sphere Satellites:

Starting from AuNSs or AuNCs adsorbed on glass substrate, asymmetric sphere core-sphere satellites (Yoon, J. H. et al., ACS Nano 2012, 6, 7199-7208) and asymmetric cube core-sphere satellites can be made. To get these assemblies, it is only necessary to modify step 2 of FIG. 6 (the second NPs are just replaced with small-sized sphere NPs). In this experiment, citrate-capped AuNPs in water phase were used (14.2±11.1 nm, 2.17 nM in 5 mL, the satellite attachment time of 12 h). Regardless of the solvent kinds, negatively charged citrate-capped AuNP does not interact with glass substrate. In the case of citrate-capped AuNP, organic solvent and NaBr are not necessary because citrate is easily replaced by Au—S bond without any treatments.

SEM images of the asymmetric AuNS core-AuNP satellites and the asymmetric AuNC core-AuNP satellites thus obtained are shown in FIGS. 12 and 13, respectively.

2. Measurements

Electron microcopy images: were taken using transmission electron microscopy (TEM) (EM 910, Zeiss) and scanning electron microscopy (SEM) (JSM-7500F, JEOL).

Extinction spectra: of samples before sonication in FIG. 6 were measured with a UV-vis absorption spectrometer (Lambda 950, Perkin Elmer).

Single-particle dark-field scattering spectra: of monomers or dimers dropped on quartz plate were obtained on a home-built setup. An inverted optical microscope (Eclipse Ti-S, Nikon) was equipped with a tungsten-halogen lamp, an oil immersion dark-field condenser (NA: 1.20-1.43), and a 100× Plan Achromat objective (NA: 0.90). For permitting only the scattered light from the targeted particle, an iris was placed in front of the spectrometer (QE Pro, Ocean Optics). When getting polarization-dependent spectra, a polarizer was introduced in front of the iris. All background-subtracted spectra were divided by the lamp spectrum for correction and then smoothed via a Savitzky-Golay filter. In this work, corrected spectra and smoothed spectra are presented as overlapped.

3. Simulations

A three-dimensional simulation dimer model was designed in the FDTD Solutions developed by Lumerical Solution, Inc. The size of gold spheres constituting the simulation dimer and the gap distance were taken from the averaged diameter (50 nm) of AuNSs in FIG. 5B and the literature (C6: 1.12 nm, C8: 1.34 nm, and C10: 1.56 nm), respectively. The frequency-dependent dielectric function of gold was taken from polynomial fitting of the experimental data obtained by Johnson and Christy. A linearly polarized total-field scattered-field (TFSF) plane wave source (400-900 nm) was employed to simulate the absorption and scattering cross sections of the dimer surrounded by a medium with an effective refractive index of 1.55. In order to determine the extinction cross section, two orthogonal TFSF was separately injected on each side of the override region (0.5 nm mesh) and then detected all absorption and scattering cross sections were averaged.

### Example 2: Symmetric Core-Satellite Assemblies (Synthesized in Suspension)

The symmetric core-satellite assembly method comprises the following two steps:

Step 1: MUTAB Functionalization on Core AuNSs

500 μL of a AuNS solution (52.9±1.6 nm, 290 pM), 120 μL of MUTAB (1.1 mM in MeCN), and 7.7 μL of NaBr (250 mM in water) were successively added into 1200 μL of MeCN. The mixture was kept for 3 h and then chemicals in the mixture were separated by centrifugation twice (850 rcf, 15 min). The precipitate was redispersed in 500 μL of D.I. (deionized) water.

Step 2: Assembly of Symmetric Sphere Core-Sphere Satellites

The prepared MUTAB-capped AuNS (290 pM, 100 μL) was added to 100-times molar excess of citrate-capped AuNPs. Keeping the molar excess, the size of citrate-capped AuNPs was varied (17, 24, and 30 nm). The color of the colloid changed in a few seconds. After 30 min, the mixture was centrifuged (850 rcf, 15 min) to get rid of the citrate-capped AuNPs unbound on MUTAB-capped AuNSs. The precipitate was redispersed in 400 μL of water.

Characterizaton

U-vis extinction spectra and TEM images of the prepared core-satellite assemblies are shown in FIGS. 10 and 11.

### Example 3: SERS Intensity of Dimers

The SERS intensity of a dimer according to the invention has been confirmed to be strong.

FIG. 18 shows the comparison of the SERS brightness from monomer and dimer suspension. Due to a large extinction (scattering+absorption) coefficient of noble metal NP, strong Rayleigh light (elastic scattering) is seen from both monomer and dimer. However, the different ability of the electromagnetic field enhancement, the inventors can see bright dot in Raman (inelastic scattering) channel only from dimers. SERS intensity from monomer is below the detection limit.

### Example 4: DNA Functionalization on Gold Nanospheres (DNA-AuNSs)

This example demonstrates the functionalization of AuNS with DNA in accordance with the present invention, as also illustrated in step a″ of FIG. 16.

### Experiment

1) DNA Functionalization

Test: As shown in the table below, 10 μL of the AuNS stock solution was diluted in the mixture of MeCN 250 μL and water 250 μL. Successively, NaBr (1.6 μL, 250 mM) was added to the prepared AuNS solution, followed by the addition of thiolated DNA (1.5 μL, 0.1 mM, HS-hexane-CCCTCCCAGTGTGGGAACAAACGGAAATAATCGAAACACCAC-3′). After gentle inversion for 5 s, the mixture was kept undisturbed at room temperature. After 1 h, it was centrifuged (600 rcf, 10 min) and redispersed in 50 μL of water.

| | vol (μL) | conc (M) | mol | ratio | final conc |
|---|---|---|---|---|---|
| HS-DNA | 1.5 | 1.00E−04 | 1.50E−10 | 1415 | 0.29 μM |
| AuNS | 10.0 | 1.06E−08 | 1.06E−13 | 1 | 0.21 nM |
| water | 250.0 | | | | |
| MeCN | 250.0 | | | | |
| NaBr | 1.6 | 0.250 | 4.00E−07 | | 779.6 μM |
| Final vol | 513.1 | | 51.3% | 48.7% | = H2O•MeCN |

Control A: MeCN, NaBr, and thiolated DNA were replaced by water and then the same procedure as used in the test group was conducted.

Control B: MeCN and NaBr were replaced by water and then the same procedure as used in the test group was conducted.

2) Gel Electrophoresis to Check the Mobility of AuNS on gel

AuNS solutions from the test, control A, and control B groups were loaded in the well of agarose gel (0.8%) immersed in TAE buffer to run gel electrophoresis (100 V, 60 min).

Results

AuNS from the test, control A, and control B group ran on lane 1, 2, and 3 on the gel, respectively, as also shown in FIG. 19. From here, AuNSs from each group are called AuNS1, AuNS2, and AuNS3. AuNS2 do not run due to the lack of negative charge on its surface. Remind that AuNS2 was prepared in the DNA-free condition. In order to run AuNS on gel, AuNS must be functionalized with at least 1 DNA. When the DNA number on AuNS increases, the AuNS mobility decreases (J. Am. Chem. Soc. 2008, 130, 2750; Nano Lett. 2011, 11, 5060). The observed mobility difference between AuNS1 and AuNS3 is clearly due to the difference of DNA number on AuNS. Thus, it has been concluded that AuNS1 is functionalized with much more DNAs than AuNS3. This result supports that the combination of organic solvent and salt are essential for the efficient CTA⁺ bilayer removal. This result shows that the condition developed in accordance with the present invention is applicable to prepare NPs functionalized with bio-molecules (such as, e.g., DNA).

### Example 5: Reproducibility of NP Assembly in Suspension

The assembly process in suspension is faster than the assembly on substrate but it might not be under control. Hence, the reproducibility of symmetric core-satellite assemblies was tested by using UV-vis spectroscopy (see FIG. 20). The measured mean extinction, mean λₘₐₓ, and mean concentration of assemblies are 0.771±0.007 (relative standard deviation, RSD=0.9%), 601.7±0.5 (RSD=0.1%) nm, and 30.1±0.1 (RSD=0.3%) pM, respectively. These small RSD values (<1%) validate the reproducibility of symmetric core-satellite particles.

### Example 6: SERS Active Core-Satellite Assemblies

Functionalizing nanostructures with Raman-active molecules gives rise to the SERS activity on it. Satellites of a core-satellite are capped with citrate molecules. And the citrate molecules not interacted with MUTAB on a core can be replaced by thiolated Raman-active molecules. For functionalizing satellites, prepared core-satellites were incubated in a 5 mM ethanolic solution of 4-nitrothiophenol (NTP) for 1 h.

For higher SERS activity, Raman-active molecules should be in the gap between a core and satellites. In order to insert NTP molecules in the gap, MUTAB functionalized cores were treated with 5 μL of a 5 mM ethanolic NTP solution for 15 min prior to the assembly. The incubation time can vary for 100 min to control the density of thiolated Raman-active molecules which replace MUTAB on the core surface.

FIG. 21 shows spectral differences of core-satellite assemblies whose Raman-active molecules are on satellites or in the gap. The core-satellite assemblies having Raman-active molecules in the gap exhibit a similar UV-vis spectrum but a six times higher SERS intensity compared to the core-satellite assemblies having Raman-active molecules on satellites. The similarity in UV-vis spectra means that core-satellites keep a similar structural property regardless of the place of NTP molecules. The higher SERS activity is due to the stronger electric field enhancement in the gap. Even though the number of adsorbed molecules is supposed to be lower in the gap than on the satellites, the extremely enhanced localized electric field in the gap leads to a higher overall SERS activity.

Any molecule which fulfills the following conditions can be used as a Raman-active molecule on the core-satellite assemblies: 1) surface seeking group to adsorb on the satellite or core surface; 2) high Raman cross section; 3) coadsorption on a core surface together with MUTAB. Eight different Raman-active molecule candidates were tested using core-satellite assemblies having them in the gap (see FIG. 22). Specifically, the following Raman-active molecules were tested: 4-nitrothiophenol (NTP), 7-mercapto-4-methylcoumarin (MMC), thio-2-naphthol (TN), 2,3,5,6-tetrafluoro-4-mercaptobenzoic acid (TFMBA), mercapto-4-methyl-5-thioacetic acid (MMTA), 2-bromo-4-mercaptobenzoic acid (BMBA), ethyl(2E,4E,6E,8E,10E,12E,14E)-15-(4-(tert-butylthio)phenyl)pentadeca-2,4,6,8,10,12,14-heptanoate (Polyene 7DB), and ethyl(2E,4E)-5-(4-(tert-butylthio)phenyl)penta-2,4-dienoate (Polyene 2DB). The observed SERS spectra indicate that all candidates are in the gap (see FIG. 22). The differences in the Raman intensity can be explained by the different Raman cross sections of the molecules and the different surface affinities leading to a different molecular population in the gap.

### Example 7: Synthesis of Quasi-Ideal Core-Satellite Assemblies

The assembly of quasi-ideal core particles and non-ideal satellite particles has been discussed above. To achieve a higher homogeneity of the core-satellite assemblies, quasi-Ideal satellite particles capped with a CTA⁺ bilayer were used instead of non-ideal satellite particles capped with citrate. Since the CTA⁺ bilayer gives positive surface charge on the particles, it must be replaced by capping agent like 11-mercaptoundecanoic acid (MUA) and 3-mercaptopropionic acid (MPA) having negative charge for core-satellite assembly. Following process is the description of the CTA⁺ bilayer removal using MUA or MPA.

1 mL of a 0.6% (mass-%) ethanolic polyvinylpyrrolidone (M_{w}≈40000 g/mol) solution was added together with 40 μL of a 5 mM MUA (or MPA) solution in a 1.5 mL tube. Afterwards, 20 μL of a 15 nM aqueous quasi-Ideal AuNP (diameter of 25 nm) suspension was rapidly added to the prepared solution and incubated at room temperature for 12 h. The mixture was centrifuged and redispersed in 100 μL of ethanol. It was performed twice to get rid of the unbound molecules and finally the redispersed AuNP solution was diluted with 600 μL of D.I. (deionized) water. The zeta-potential value of the diluted AuNP solution was measured at −12.08±3.5 mV. It indicates all CTA⁺ molecules are replaced by MUA.

Indeed, a core and satellites are distanced by MUTAB on core and MUA (or MPA) on satellite. The calculated gap distances are 2.5 nm (for MPA; HS—C₂—COOH) and 3.4 nm (for MUA; HS—C₁₀—COOH). Thus, the core-satellite whose satellites are functionalized with MPA is expected to have a smaller gap leading to red-shifted SP coupling band. This is confirmed in the UV-vis spectra shown in FIG. 23. The gap difference of 0.9 nm induces 20 nm difference in SP coupling band position. This fact implies that HS—Cₙ—COOH (n=3, 4, 5, 6, 7, 8, and 9) can be exploited to control the SP coupling energy. The SEM images in FIG. 23 show that the morphology of the quasi-ideal core-satellite assemblies is highly uniform in terms of roundness of the constituent particles. This uniformity will be beneficial to quantitative studies at single particle level.

1. A method of preparing a dimeric nanoparticle assembly, the method comprising:
(i) contacting a first metal nanoparticle (NP1), having a bilayer of a long-chained cationic quaternary ammonium compound bound to its surface, with a negatively charged substrate to obtain NP1 bound to the surface of the negatively charged substrate;
(ii) subjecting NP1, which is bound to the surface of the negatively charged substrate via the bilayer of the long-chained cationic quaternary ammonium compound, to an alkali metal or alkaline earth metal halide and a polar organic solvent to remove the bilayer of the long-chained cationic quaternary ammonium compound from those parts of the surface of NP1 that are not bound to the surface of the negatively charged substrate;
(iii) subjecting NP1, which is bound to the surface of the negatively charged substrate via the bilayer of the long-chained cationic quaternary ammonium compound, to a compound HS—R—X and a polar organic solvent to allow the formation of a self-assembled monolayer of the compound HS—R—X on those parts of the surface of NP1 that are not bound to the negatively charged substrate, wherein R is an organic group and X is a functional group containing a sulfur atom or a nitrogen atom;
(iv) contacting NP1, which is bound to the surface of the negatively charged substrate via the bilayer of the long-chained cationic quaternary ammonium compound and which has a self-assembled monolayer of the compound HS—R—X bound to those parts of its surface that are not bound to the negatively charged substrate, with a polar organic solvent, an alkali metal or alkaline earth metal halide and a second metal nanoparticle (NP2), wherein NP2 has a bilayer of a long-chained cationic quaternary ammonium compound bound to its surface, to obtain a conjugate of NP1 and NP2, wherein NP1 and NP2 are linked together in said conjugate via a part of the self-assembled monolayer of the compound HS—R—X, said part being bound to the metal surface of both NP1 and NP2, wherein said conjugate of NP1 and NP2 is bound to the surface of the negatively charged substrate via the bilayer of the long-chained cationic quaternary ammonium compound that is bound to the surface of NP1; and
(v) subjecting the conjugate of NP1 and NP2, which is bound to the surface of the negatively charged substrate via the bilayer of the long-chained cationic quaternary ammonium compound that is bound to the surface of NP1, and which has a bilayer of the long-chained cationic quaternary ammonium compound bound to the surface of NP2, to a compound containing an N,N,N-trialkylammonium group and/or a thiol group, an alkali metal or alkaline earth metal halide and a polar organic solvent to remove the bilayer of the respective long-chained cationic quaternary ammonium compound from both NP1 and NP2, allow the formation of a self-assembled monolayer of the compound containing an N,N,N-trialkylammonium group and/or a thiol group on those parts of the surface of both NP1 and NP2 that are not bound by the self-assembled monolayer of the compound HS—R—X, and release the conjugate of NP1 and NP2 from the surface of the negatively charged substrate to provide the dimeric nanoparticle assembly,
wherein the dimeric nanoparticle assembly thus obtained comprises NP1 and NP2, wherein NP1 comprised in the dimeric nanoparticle assembly has a self-assembled monolayer of the compound containing an N,N,N-trialkylammonium group and/or a thiol group bound to one part of its surface and a self-assembled monolayer of the compound HS—R—X bound to the remaining part of its surface, wherein NP1 and NP2 are linked together via a part of the self-assembled monolayer of the compound HS—R—X, which part is bound to the surface of both NP1 and NP2, and wherein NP2 comprised in the dimeric nanoparticle assembly has a self-assembled monolayer of the compound containing an N,N,N-trialkylammonium group and/or a thiol group bound to the part of its surface that is not bound by the self-assembled monolayer of the compound HS—R—X.

2. The method of claim 1, wherein the compound HS—R—X is an alkanedithiol, preferably a compound HS—(CH₂)₂₋₂₀—SH, more preferably a compound HS—(CH₂)₆₋₁₁—SH, or even more preferably a compound selected from 1,8-hexanedithiol, 1,8-octanedithiol and 1,10-decanedithiol.

3. The method of claim 1 or 2, wherein the compound containing an N,N,N-trialkylammonium group and/or a thiol group is an N,N,N-trialkylammonium-substituted thiol compound, preferably an N,N,N-tri(C₁₋₄ alkyl)ammonium-alkanethiol, more preferably a compound N⁺(C₁₋₄ alkyl)₃-(C₂₋₁₆ alkylene-SH, even more preferably a compound N⁺(CH₃)₃—(CH₂)₂₋₁₆—SH, yet even more preferably a compound N⁺(CH₃)₃—(CH₂)₁₁—SH.

4. The method of any one of claims 1 to 3, wherein the long-chained cationic quaternary ammonium compound that is bound to the surface of NP1 is an (N,N,N-trialkyl)alkylammonium compound or an alkylpyridinium compound, preferably a compound (C₈₋₂₂ alkyl)-N⁺(C₁₋₄ alkyl)₃ or a (C₈₋₂₂ alkyl)-pyridinium compound, more preferably a compound (C₈₋₂₂ alkyl)-N⁺(CH₃)₃, even more preferably a compound H₃C—(CH₂)₇₋₂₁—N⁺(CH₃), yet even more preferably a compound H₃C—(CH₂)₁₅—N⁺(C H₃)₃.

5. The method of any one of claims 1 to 4, wherein the long-chained cationic quaternary ammonium compound that is bound to the surface of NP2 is an (N,N,N-trialkyl)alkylammonium compound or an alkylpyridinium compound, preferably a compound (C₈₋₂₂ alkyl)-N⁺(C₁₋₄ alkyl) or a (C₈₋₂₂ alkyl)-pyridinium compound, more preferably a compound (C₈₋₂₂ alkyl)-N⁺(CH₃)₃, even more preferably a compound H₃C—(CH₂)₇₋₂₁—N⁺(CH₃)₃ yet even more preferably a compound H₃C—(CH₂)₁₅—N⁺(CH₃)₃.

6. The method of any one of claims 1 to 5, wherein the long-chained cationic quaternary ammonium compound that is bound to the surface of NP1 and the long-chained cationic quaternary ammonium compound that is bound to the surface of NP2 are the same.

7. The method of any one of claims 1 to 6, wherein the first metal nanoparticle is a noble metal nanoparticle, preferably a gold nanoparticle or a silver nanoparticle, more preferably a gold nanoparticle.

8. The method of any one of claims 1 to 7, wherein the first metal nanoparticle is a spherical or a cubic nanoparticle, preferably a spherical nanoparticle.

9. The method of any one of claims 1 to 8, wherein the first metal nanoparticle is a spherical nanoparticle, and further wherein: at least about 90 mol-% of the first metal nanoparticle has a roundness value of at least about 0.94, and/or the relative standard deviation in the particle size distribution of the first metal nanoparticle is smaller than about 6.0%.

10. The method of any one of claims 1 to 9, wherein the second metal nanoparticle is a noble metal nanoparticle, preferably a gold nanoparticle or a silver nanoparticle, more preferably a gold nanoparticle.

11. The method of any one of claims 1 to 10, wherein the second metal nanoparticle is a spherical or a cubic nanoparticle, preferably a spherical nanoparticle.

12. The method of any one of claims 1 to 11, wherein the second metal nanoparticle is a spherical nanoparticle, and further wherein at least about 90 mol-% of the second metal nanoparticle has a roundness value of at least about 0.94, and/or the relative standard deviation in the particle size distribution of the second metal nanoparticle is smaller than about 6.0%.

13. The method of any one of claims 1 to 12, wherein the first metal nanoparticle is subjected to chemical etching before it is used in step (i), and/or wherein the second metal nanoparticle is subjected to chemical etching before it is used in step (iv).

14. The method of any one of claims 1 to 13, wherein the first and the second metal nanoparticle each have a particle size of at least about 50 nm.

15. The method of any one of claims 1 to 14, wherein each one of the first and the second metal nanoparticle is a spherical gold nanoparticle having a diameter of at least about 50 nm, and wherein the two nanoparticles preferably have essentially the same diameter.

16. The method of any one of claims 1 to 15, wherein the negatively charged substrate is a glass substrate.

17. The method of any one of claims 1 to 16, wherein the alkali metal or alkaline earth metal halide used in step (I), step (iv) and/or step (v) is independently selected from sodium bromide, sodium chloride, potassium bromide and potassium chloride, wherein it is preferably sodium bromide or sodium chloride, more preferably sodium bromide.

18. The method of any one of claims 1 to 17, wherein the polar organic solvent used in step (ii), step (Iii), step (iv) and/or step (v) is independently selected from an alcohol, dimethylformamide, dimethyl sulfoxide, acetone, acetonitrile, and a mixture of any one of the aforementioned polar organic solvents with water, wherein it is preferably ethanol or acetonitrile.

19. The method of any one of claims 1 to 18, wherein step (i) is conducted in an aqueous solution of the long-chained cationic quaternary ammonium compound, wherein the concentration of the long-chained cationic quaternary ammonium compound in said aqueous solution is preferably about 1.5 μM to about 10 μM.

20. The method of any one of claims 1 to 19, wherein step (iii) comprises subjecting NP1, which is bound to the surface of the negatively charged substrate via the bilayer of the long-chained cationic quaternary ammonium compound, to a compound HS—R—X, a compound HS—R and a polar organic solvent to allow the formation of a self-assembled monolayer of the compounds HS—R—X and HS—R on those parts of the surface of NP1 that are not bound to the negatively charged substrate, wherein the group R comprised in the compound HS—R—X and in the compound HS—R is independently an organic group and wherein the group X comprised in the compound HS—R—X is a functional group containing a sulfur atom or a nitrogen atom.

21. The method of any one of claims 1 to 20, wherein steps (ii) and (ii) are conducted simultaneously by subjecting NP1, which is bound to the surface of the negatively charged substrate via the bilayer of the long-chained cationic quaternary ammonium compound, to an alkali metal or alkaline earth metal halide, a compound HS—R—X and a polar organic solvent to remove the bilayer of the long-chained cationic quaternary ammonium compound from those parts of the surface of NP1 that are not bound to the surface of the negatively charged substrate and to allow the formation of a self-assembled monolayer of the compound HS—R—X on those parts of the surface of NP1.

22. The method of any one of claims 1 to 21, wherein in step (iv) the alkali metal or alkaline earth metal halide is used in a concentration of about 100 μM to about 300 μM.

23. The method of any one of claims 1 to 22, wherein in step (v) the release the conjugate of NP1 and NP2 from the surface of the negatively charged substrate is facilitated by using sonication.

24. The method of any one of claims 1 to 23, comprising a further step of coupling a binding molecule to the dimeric nanoparticle assembly.

25. The method of claim 24, wherein the binding molecule is an antibody or an antigen-binding fragment thereof.

26. A dimeric nanoparticle assembly obtainable by the method of any one of claims 1 to 25.

27. A method of preparing a core-satellite nanoparticle assembly, the method comprising:
(i) subjecting a first metal nanoparticle (NP1), having a bilayer of a long-chained cationic quaternary ammonium compound bound to its surface, to a compound containing an N,N,N-trialkylammonium group and a thiol group, an alkali metal or alkaline earth metal halide and a polar organic solvent to remove the bilayer of the long-chained cationic quaternary ammonium compound from the surface of NP1 and to allow the formation of a self-assembled monolayer of the compound containing an N,N,N-trialkylammonium group and a thiol group on the surface of NP1; and
(ii) contacting NP1, which has a self-assembled monolayer of the compound containing an N,N,N-trialkylammonium group and a thiol group on its surface, with a molar excess of negatively charged nanoparticles to obtain the core-satellite nanoparticle assembly,
wherein the core-satellite nanoparticle assembly thus obtained comprises NP1 having a self-assembled monolayer of the compound containing an N,N,N-trialkylammonium group and a thiol group bound to its surface, and wherein the negatively charged nanoparticles are bound to the outer surface of the self-assembled monolayer of the compound containing an N,N,N-trialkylammonium group and a thiol group.

28. The method of claim 27, wherein the long-chained cationic quaternary ammonium compound that is bound to the surface of NP is an (N,N,N-trialkyl)alkylammonium compound or an alkylpyridinium compound, preferably a compound (C₈₋₂₂-alkyl)-N⁺(C₁₋₄ alkyl)₃ or a (C₈₋₂₂ alkyl)-pyridinium compound, more preferably a compound (C₈₋₂₂ alkyl)-N⁺(CH₃)₃, even more preferably a compound H₃C—(CH₂)₇₋₂₁—N⁺(CH₃)₃, yet even more preferably a compound H₃C—(CH₂)₁₅—N⁺(CH₃)₃.

29. The method of claim 27 or 28, wherein the compound containing an N,N,N-trialkylammonium group and a thiol group is an N,N,N-tri(C₁₋₄ alkyl)ammonium-alkanethiol, preferably a compound N⁺(C₁₋₄ alkyl)₃-(C₂₋₁₆ alkylene)-SH, more preferably a compound N⁺(CH₃)₃—(CH₂)₂₋₁₆—SH, even more preferably a compound N⁺(CH₃)₃—(CH₂)₁₁—SH.

30. The method of any one of claims 27 to 29, wherein the alkali metal or alkaline earth metal halide is selected from sodium bromide, sodium chloride, potassium bromide and potassium chloride, wherein it is preferably sodium bromide or sodium chloride, more preferably sodium bromide.

31. The method of any one of claims 27 to 30, wherein the polar organic solvent is selected from an alcohol, dimethylformamide, dimethyl sulfoxide, acetone, acetonitrile, and a mixture of any one of the aforementioned polar organic solvents with water, wherein the polar organic solvent is preferably ethanol or acetonitrile.

32. The method of any one of claims 27 to 31, wherein the first metal nanoparticle is a noble metal nanoparticle, preferably a gold nanoparticle or a silver nanoparticle, more preferably a gold nanoparticle.

33. The method of any one of claims 27 to 32, wherein the first metal nanoparticle is a spherical or a cubic nanoparticle, preferably a spherical nanoparticle.

34. The method of any one of claims 27 to 33, wherein the first metal nanoparticle is a spherical nanoparticle, and further wherein: at least about 90 mol-% of the first metal nanoparticle has a roundness value of at least about 0.94, and/or the relative standard deviation in the particle size distribution of the first metal nanoparticle is smaller than about 6.0%.

35. The method of any one of claims 27 to 34, wherein the first metal nanoparticle is subjected to chemical etching before it is used in step (i).

36. The method of any one of claims 27 to 35, wherein the first metal nanoparticle has a particle size of at least about 50 nm.

37. The method of any one of claims 27 to 36, wherein in step (i) NP1 is contacted with at least a 50-fold molar excess of the negatively charged nanoparticles, preferably with at least a 100-fold molar excess of the negatively charged nanoparticles.

38. The method of any one of claims 27 to 37, wherein the negatively charged nanoparticles are citrate-capped metal nanoparticles, preferably citrate-capped gold or silver nanoparticles.

39. The method of any one of claims 27 to 38, wherein the negatively charged nanoparticles have a particle size that is ⅕ or less of the particle size of NP1, preferably 1/10 or less of the particle size of NP1, more preferably 1/50 or less of the particle size of NP1, even more preferably 1/100 or less of the particle size of NP1.

40. The method of any one of claims 27 to 39, wherein the negatively charged nanoparticles are spherical or cubic nanoparticles, preferably spherical nanoparticles.

41. A core-satellite nanoparticle assembly obtainable by the method of any one of claims 27 to 40.

42. A method of preparing a functionalized nanoparticle, the method comprising:
(i) contacting a first metal nanoparticle (NP1), having a bilayer of a long-chained cationic quaternary ammonium compound bound to its surface, with a negatively charged substrate to obtain NP1 bound to the surface of the negatively charged substrate;
(ii) subjecting NP1, which is bound to the surface of the negatively charged substrate via the bilayer of the long-chained cationic quaternary ammonium compound, to an alkali metal or alkaline earth metal halide and a polar organic solvent to remove the bilayer of the long-chained cationic quaternary ammonium compound from those parts of the surface of NP1 that are not bound to the surface of the negatively charged substrate;
(iii) subjecting NP1, which is bound to the surface of the negatively charged substrate via the bilayer of the long-chained cationic quaternary ammonium compound, to a thiolated biomolecule and a polar organic solvent to allow the formation of a self-assembled monolayer of the thiolated biomolecule on those parts of the surface of NP1 that are not bound to the negatively charged substrate; and
(iv) subjecting NP1, which is bound to the surface of the negatively charged substrate via the bilayer of the long-chained cationic quaternary ammonium compound and which has a self-assembled monolayer of the thiolated biomolecule bound to those parts of its surface that are not bound to the negatively charged substrate, to a thiolated biomolecule, an alkali metal or alkaline earth metal halide and a polar organic solvent to remove the bilayer of the long-chained cationic quaternary ammonium compound from NP1, allow the formation of a self-assembled monolayer of the thiolated biomolecule on those parts of the surface of NP1 from which the bilayer of the long-chained cationic quaternary ammonium compound is removed, and release NP1 having a self-assembled monolayer of the respective thiolated biomolecule bound to its surface from the surface of the negatively charged substrate to provide the functionalized nanoparticle.

43. The method of claim 42, wherein the long-chained cationic quaternary ammonium compound that is bound to the surface of NP1 is an (N,N,N-trialkyl)alkylammonium compound or an alkylpyridinium compound, preferably a compound (C₈₋₂₂ alkyl)-N⁺(C₁₋₄ alkyl)₃ or a (C₈₋₂₂ alkyl)-pyridinium compound, more preferably a compound (C₈₋₂₂ alkyl)-N⁺(CH₃)₃, even more preferably a compound H₃C—(CH₂)₇₋₂₁—N⁺(CH₃)₃, yet even more preferably a compound H₃C—(CH₂)₁₅—N⁺(CH₃)₃.

44. The method of any claim 42 or 43, wherein the first metal nanoparticle is a noble metal nanoparticle, preferably a gold nanoparticle or a silver nanoparticle, more preferably a gold nanoparticle.

45. The method of any one of claims 42 to 44, wherein the first metal nanoparticle is a spherical or a cubic nanoparticle, preferably a spherical nanoparticle.

46. The method of any one of claims 42 to 45, wherein the first metal nanoparticle is a spherical nanoparticle, and further wherein: at least about 90 mol-% of the first metal nanoparticle has a roundness value of at least about 0.94, and/or the relative standard deviation in the particle size distribution of the first metal nanoparticle is smaller than about 6.0%.

47. The method of any one of claims 42 to 46, wherein the first metal nanoparticle is subjected to chemical etching before it is used in step (i).

48. The method of any one of claims 42 to 47, wherein the first metal nanoparticle has a particle size of at least about 50 nm.

49. The method of any one of claims 42 to 48, wherein the negatively charged substrate is a glass substrate.

50. The method of any one of claims 42 to 49, wherein the alkali metal or alkaline earth metal halide used in step (ii) and/or step (iv) is independently selected from sodium bromide, sodium chloride, potassium bromide and potassium chloride, wherein it is preferably sodium bromide or sodium chloride, more preferably sodium bromide.

51. The method of any one of claims 42 to 50, wherein the polar organic solvent used in step (i), step (II) and/or step (iv) is independently selected from an alcohol, dimethylformamide, dimethyl sulfoxide, acetone, acetonitrile, and a mixture of any one of the aforementioned polar organic solvents with water, wherein it is preferably ethanol or acetonitrile.

52. The method of any one of claims 42 to 51, wherein the thiolated biomolecule used in step (ii) and/or step (iv) is independently a thiolated nucleic acid, preferably a thiolated DNA.

53. The method of any one of claims 42 to 52, wherein the thiolated biomolecule used in step (iii) and the thiolated biomolecule used in step (iv) are the same.

54. The method of any one of claims 42 to 53, wherein step (i) is conducted in an aqueous solution of the long-chained cationic quaternary ammonium compound, wherein the concentration of the long-chained cationic quaternary ammonium compound in said aqueous solution is preferably about 1.5 μM to about 10 μM.

55. The method of any one of claims 42 to 54, wherein steps (ii) and (iii) are conducted simultaneously by subjecting NP1, which is bound to the surface of the negatively charged substrate via the bilayer of the long-chained cationic quaternary ammonium compound, to an alkali metal or alkaline earth metal halide, a thiolated biomolecule and a polar organic solvent to remove the bilayer of the long-chained cationic quaternary ammonium compound from those parts of the surface of NP1 that are not bound to the surface of the negatively charged substrate and to allow the formation of a self-assembled monolayer of the thiolated biomolecule on those parts of the surface of NP1.

56. The method of any one of claims 42 to 55, wherein in step (iv) the release of NP1 having a self-assembled monolayer of the thiolated biomolecule bound to its surface from the surface of the negatively charged substrate is facilitated by using sonication.

57. A functionalized nanoparticle obtainable by the method of any one of claims 42 to 56.

58. Use of the dimeric nanoparticle assembly of claim 26 or the core-satellite nanoparticle assembly of claim 41 or the functionalized nanoparticle of claim 57 as a marker in plasmonic spectroscopy, preferably as a surface-enhanced Raman scattering (SERS) marker.

59. A plasmonic spectroscopy marker comprising the dimeric nanoparticle assembly of claim 26 or the core-satellite nanoparticle assembly of claim 41 or the functionalized nanoparticle of claim 57.

60. A surface-enhanced Raman scattering (SERS) marker comprising the dimeric nanoparticle assembly of claim 26 or the core-satellite nanoparticle assembly of claim 41 or the functionalized nanoparticle of claim 57.
